(19) United States
(12) Patent Application Publication
(10) Pub. No.: US 2021/0048686 A1
(43) Pub. Date: Feb. 18, 2021

(54) ADJUSTMENT OF THE SUBJECTIVE AND OBJECTIVE REFRACTIONS

(71) Applicant: Rodenstock GmbH, Munchenü (DE)

(72) Inventors: Adam Muschielok, Muenchen (DE); Helmut Altheimer, Baisweil-Lauchdorf (DE); Wolfgang Becken, Neuried (DE); Gregor Esser, Muenchen (DE); Dietmar Uttenweiler, Icking (DE)

(21) Appl. No.: 17/043,043

(22) PCT Filed: Mar. 28, 2019
(86) PCT No.: PCT/EP2019/057820

§ 371 Date: Sep. 29, 2020

(30) Foreign Application Priority Data
Mar. 29, 2018 (DE) 10 2018 002 630.3

(51) Int. Cl. G02C 7/02; G16H 50/30; G16H 50/20; G16H 50/70; G06N 7/00; A61B 3/103; A61B 3/00; A61B 3/028

(52) U.S. Cl. CPC G02C 7/027; G16H 50/30; G16H 50/20; G16H 50/70; G02C 2202/22; A61B 3/103; A61B 3/0025; A61B 3/028; G06N 7/00

(57) ABSTRACT

A method for determining the defective vision of an eye of a spectacle wearer, corresponding computer program products, spectacle glass production methods and devices. Also, a spectacle glass or a spectacle glass series. The method for determining the defective vision of an eye of a spectacle wearer includes: providing measurement values from a first and a second measurement of the defective vision of the eye of the spectacle wearer; and calculating an estimated value for the defective vision of the eye of the spectacle wearer on the basis of the measurement values from the first and the second measurement, measurement inaccuracies from the first and the second measurements of the defective vision being taken into account in the calculation of the estimated value of the defective vision.

### TECHNICAL FIELD

The present invention relates to methods for determining the vision disorder of a spectacle wearer, corresponding computer program products, spectacle lens manufacturing methods and devices. The present invention also relates to a spectacle lens or a spectacle lens series.

### BACKGROUND

A widely used method for determining a refraction (comprising at least one refraction component) is the so-called subjective refraction determination, which has become generally accepted among opticians. In the subjective refraction determination, different refraction lenses are conventionally presented to the wearer of a spectacle lens, wherein the wearer of the spectacle lens informs the refractionist about an improvement or deterioration in the visual impression upon change of the optical properties of the refraction lens presented. The subjective refraction thus requires information from the tested person about the visual impression and can also take into account the influence of other variables on the visual impression.

The subjective refraction determination can, for example, build on values of an objective refraction determination or on values of spectacles already worn. However, the accuracy of the subjective refraction determination depends critically on the skill of the refractionist, for example an optician and/or an ophthalmologist who carries out the subjective refraction determination. The subjective refraction determination also depends critically on the person to be tested, in particular on the ability of the person to be tested to assess and/or articulate the sharpness of the visual impressions.

Another method for determining a refraction is the so-called objective refraction: The objective refraction is carried out using an apparatus arrangement and is determined by the refractive properties and the geometry of the eyeball. The objective refraction can be carried out using various devices, such as refractometer, aberrometer, wavefront scanner, etc.

Frequently, however, the values of a spectacle wearer determined using objective refraction differ considerably from the values determined using subjective refraction. This makes it much more difficult to find suitable target values for the spectacle lens, which are to correct the vision disorder of the spectacle wearer.

WO 2009/007136 A1 describes a method for determining target values for a spectacle lens, in which at least a subset of the subjective refraction data is adapted to the objective refraction data based on a comparison of the subjective and objective data. In particular, the subset of the subjective refraction data is adapted to the objective refraction data if the comparison result satisfies at least one predetermined comparison condition, otherwise the subset of the subjective refraction data is maintained.

### SUMMARY

It is an object of the present invention to improve the determination of the vision disorder of a spectacle wearer. This object is achieved with the methods, devices, computer program products, spectacle lenses and spectacle lens series according to the independent claims. Preferred variants or embodiments are the subject of the dependent claims.

The present invention is based on the finding that different measurements or measuring methods and/or measuring devices basically deliver different refraction values. The invention proposes taking into account measurement inaccuracies or measurement deviations of the different measurements in the calculation of the vision disorder of a spectacle wearer.

According to a first aspect of the invention, a computer-implemented or computer-aided method for determining the vision disorder of an eye of a spectacle wearer is described, the method comprising:
providing measurement values from a first and a second measurement of the vision disorder of the eye of the spectacle wearer;
calculating an estimated value for the vision disorder of the eye of the spectacle wearer based on the measurement values from the first and the second measurement, wherein measurement inaccuracies or measurement deviations of the first and the second measurements of the vision disorder are taken into account in the calculation of the estimated value of the vision disorder, or measurement inaccuracies or measurement deviations of the first measurement and the second measurement of the vision disorder are considered in the calculation of the estimated value of the vision disorder.

“Providing” within the meaning of the present invention includes “taking from a database, a table or another data carrier”, “input into a user interface, such as a graphical user interface”, “transmitting”, “measuring” or “estimating”.

The first and the second measurement can be carried out subjectively/objectively using measuring devices for measuring the vision disorder of various types, for example using different apparatuses, etc.

The measurement values can comprise measurement values of at least one component, preferably of several components. In other words, the measurement values can be in vector form with several components. The components can e.g. be
the components of a polar representation (sphere, cylinder and axis),
the components of a curvature matrix representation,
the components of a power vector representation (M, J0 and J45),
the components of a Harris vector representation,
the components of a Zernike polynomial decomposition (Zernike coefficient), or
the component of another suitable characterization of the vision disorder of a spectacle wearer.

The measurement inaccuracies or measurement deviations of the first and second measurements can be determined in advance (for example according to one of the methods described below) and stored in a suitable form (for example as a table, in a file, in a database, as a mathematical model, as a function, etc.). Accordingly, the method can comprise providing data or information about the measurement inaccuracies or measurement deviations of the first and second measurements of the vision disorder. Moreover, the method can comprise providing data about the type of measurement, the respective device used, individual data of the spectacle wearer (such as age, preferences, viewing habits, use of the spectacle lens, parameters of the wearing position of the spectacle lens, etc.).

According to a second aspect, a method for determining the target power of a spectacle lens for correcting a vision disorder of a spectacle wearer is proposed, the method comprising:
determining the vision disorder of an eye of the spectacle wearer according to the method according to the first aspect; and
setting the target power based on the determined vision disorder, so that the target power corrects the determined vision disorder at least partially, preferably substantially completely, in at least one reference point.

The reference point can be the distance reference point, the prism reference point, the centration point or the centration cross, the near reference point or another suitable reference point.

According to a third aspect, a method for producing a spectacle lens is proposed, the method comprising:
determining the vision disorder of an eye of the spectacle wearer according to the method according to the second aspect;
setting the target power in at least one reference point of the spectacle lens based on the determined vision disorder, so that the target power of the spectacle lens corrects the determined vision disorder at least partially, preferably substantially completely, in the at least one reference point; and
manufacturing the spectacle lens so that the target power is achieved in the at least one reference point of the spectacle lens, preferably in a predetermined wearing position of the spectacle lens.

According to a fourth aspect, a method for ordering spectacle lenses is proposed, comprising:
providing measurement values from a first measurement and a second measurement of the vision disorder of the eye of the spectacle wearer;
calculating an estimated value for the vision disorder of the eye of the spectacle wearer using the measurement values from the first measurement and the second measurement, wherein measurement inaccuracies or measurement deviations of the first measurement and the second measurement of the vision disorder are taken into account in the calculation of the estimated value of the vision disorder.

According to a fifth aspect, a computer program product is proposed, which, when loaded into the memory of a computer and executed on a computer, causes the computer to execute a method according to one of the above aspects.

According to a sixth aspect of the invention, a device for determining the vision disorder of an eye of a spectacle wearer with a computing device, in particular a computer or computer system, is proposed, which is designed to execute the method according to one of the above aspects.

According to a seventh aspect of the invention, a device for producing a spectacle lens is proposed, the device comprising:
a device for determining the vision disorder of an eye of a spectacle wearer according to the sixth aspect;
a device for setting the target power in a reference point of the spectacle lens on the basis of the determined vision disorder, so that the target power of the spectacle lens corrects the determined vision disorder at least partially, preferably substantially completely, in the at least one reference point; and
a manufacturing device for manufacturing the spectacle lens, so that the target power is achieved in at least one predetermined reference point of the spectacle lens, preferably in a predetermined wearing position of the spectacle lens.

According to an eighth aspect of the invention, a device for ordering spectacle lenses is proposed, which is designed to carry out the method for ordering spectacle lenses. In particular, the device for ordering spectacle lenses comprises:
a device for providing measurement values from a first measurement and a second measurement of the vision disorder of the eye of the spectacle wearer, and
a computing device designed to calculate an estimated value for the vision disorder of the eye of the spectacle wearer based on the measurement values from the first measurement and the second measurement, wherein measurement inaccuracies or measurement deviations of the first measurement and the second measurement of the vision disorder are taken into account in the calculation of the estimated value of the vision disorder. The estimated value can be determined using one of the methods described above.

The above-mentioned devices for providing, determining or setting or calculating data and/or measurement values can be realized by suitably configured or programmed data processing devices (in particular specialized hardware modules, computers or computer systems) with corresponding computing units, electronic interfaces, memories and data transmission units. The devices can further comprise at least one preferably interactive graphical user interface (GUI), which enables a user to input and/or modify data.

The manufacturing device can comprise e.g. at least one CNC-controlled machine for direct machining of a blank according to the determined optimization specifications. Alternatively, the spectacle lens can be manufactured using a casting method. Preferably, the finished spectacle lens has a simple spherical or rotationally symmetrical aspherical surface and a surface calculated or optimized according to the method according to the invention and according to individual parameters of the spectacle wearer. Preferably, the simple spherical or rotationally symmetrical aspherical surface is the front surface (i.e. the object-side surface) of the spectacle lens. It is of course possible, however, to arrange the optimized surface as the front surface of the spectacle lens. It is also possible to optimize both surfaces of the spectacle lens.

A ninth aspect of the invention relates to a spectacle lens or a series of spectacle lenses that can be produced using the proposed production method. In particular, a spectacle lens for correcting the vision disorder of the eye of a spectacle wearer is proposed, wherein:
the spectacle lens has a first power P_A in a reference point of the spectacle lens, and
the vision disorder is characterized by at least a first measurement value P_A1 obtained using a measuring device of the first type for measuring the vision disorder and consisting of several components, and at least a second measurement value P_A2 obtained using a measuring device of the second type for measuring the vision disorder and consisting of several components, the first measurement value P_A1 and the second measurement value P_A2 differing in at least one component X;
the component X of the first power P_A present in the reference point of the spectacle lens is closer to the component X of the measurement value among the measurement values P_A1 or P_A2 of the spectacle lens that is obtained from the measuring device with the lower inaccuracy in the measurement of the component X, and wherein
the components of the measurement values P_A1 and P_A2 are components of a wavefront representation of the vision disorder, its linear combination or variables derived therefrom.

The spectacle lens can be a single vision spectacle lens or a progressive spectacle lens. Also, the spectacle lenses of the series can be single vision spectacle lenses or progressive spectacle lenses.

A tenth aspect relates to a set of a spectacle lens according to the above aspect for correcting a vision disorder of a spectacle wearer and a specification assigned to the spectacle lens, the specification comprising the first measurement value P_A1 and the second measurement value P_A2. The specification can be stored on a suitable data carrier, e.g. on paper or on an electronic or optical data carrier. For example, the specification can be printed on a spectacle lens bag. The specification can also be present in or on the spectacle lens itself, e.g. by being engraved in or on the spectacle lens.

An eleventh aspect of the invention relates to a series of spectacle lenses or a series of sets of spectacle lenses and specifications assigned to the respective spectacle lenses. The lenses of the series can be the lenses described above. In particular, the series comprises:
a first spectacle lens A for correcting a vision disorder of a first eye of a spectacle wearer, wherein the spectacle lens A has a first power P_A in a reference point of the spectacle lens, wherein the vision disorder of the first eye is characterized by at least a first measurement value P_A1 obtained using a measuring device of the first type for measuring the vision disorder and consisting of several components, and at least a second measurement value P_A2 obtained using a measuring device of the second type for measuring the vision disorder and consisting of several components, wherein optionally the first measurement value P_A1 and the second measurement value P_A2 differ in at least one component X;
a second spectacle lens B for correcting a vision disorder of a second eye of a spectacle wearer, wherein the spectacle lens B has a second power P_B in a reference point identified identically in comparison with the first spectacle lens, wherein the vision disorder of the second eye is characterized by at least a first measurement value P_B1 obtained using a measuring device of the first type for measuring the vision disorder and consisting of several components, and at least a second measurement value P_B2 obtained using a measuring device of the second type for measuring the vision disorder and consisting of several components, wherein optionally the first measurement value P_B1 and the second measurement value P_B2 differ in at least one component X;
at least a third spectacle lens C for correcting a vision disorder of a third eye of a spectacle wearer, wherein the spectacle lens C has a third power P_C in a reference point identified identically in comparison with the first spectacle lens, and wherein the vision disorder of the third eye is characterized by at least a first measurement value P_C1 obtained using a measuring device of the first type for measuring the vision disorder and consisting of several components, and at least a second measurement value P_C2 obtained using a measuring device of the second type for measuring the vision disorder and consisting of several components, wherein optionally the first measurement value P_C1 and the second measurement value P_C2 differ in at least one component X;
   wherein:
the first measurement values P_A1, P_B1 and P_C1 determined with the measuring device of the first type are identical in terms of components,
the components X of the second measurement values P_A2, P_B2 and P_C2 determined with the measuring device of the second type all differ pairwise,
the component X of the first power P_A and the component X of the first measurement value P_A1 are almost identical, and
wherein for the components X of the power of the i^{th} spectacle lens present in the reference point, X_i, where i=A, B or C, and for the components X of the second measurement values of the i^{th} eyes, X_i2, the following relationships apply:

(X_B−X_A)/(X_B2−X_A2) unequal (X_C−X_A)/(X_C2−X_A2);

abs(X_B2−X_A2)<abs(X_C2−X_A2); and

signum(X_B2−X_A2)=signum(X_C2−X_A2).

Furthermore, the invention offers a use of a spectacle lens produced according to the production method according to the invention in a predetermined average or ideal wearing position of the spectacle lens in front of the eyes of a specific spectacle wearer for correcting a vision disorder of the spectacle wearer, the vision disorder being characterized by a measurement value determined using a first measuring device and a measurement value determined using a second measuring device.

The methods, devices and computer program products in accordance with one of the above aspects can reduce the probability of reclamation of spectacle lenses, in the calculation of which both the subjective and the objective refraction is used. This relates specifically to power ranges in which the apparatuses for objective refraction systematically measure differently than the subjective refraction.

### PREFERRED EXAMPLES

The measurement inaccuracies can comprise a statistical and/or a systematical deviation between the measurement values from the first measurement and the measurement values from the second measurement. If, for example, the systematical or statistical deviation between the first and second measurement is not taken into account, there may be considerable deviations in the refraction values determined, e.g. averaged, according to the prior art from the values optimal for the spectacle wearer.

According to a preferred example, the measurement inaccuracies or measurement deviations comprise both a statistical and a systematical deviation of the first measurement from the second measurement. The systematical and statistical deviations can be taken into account in a single method step or in several method steps one after the other in an arbitrary order.

In a preferred example, a first estimated value for the vision disorder of the eye of the spectacle wearer is calculated based on the first and the second measurement, wherein systematic deviations between the measurement values from the first measurement and the second measurement of the vision disorder are taken into account in the calculation of the first estimated value of the vision disorder. In a second step, a second estimated value of the vision disorder is determined based on the first estimated value and the statistical measurement inaccuracies or measurement deviations of the first and second measurements, which second estimated value is output as the final estimated value or is further adapted.

The determination of the first estimated value can comprise determining a correction term for the measurement values from the first and/or the second measurement and a correction or adaptation of the measurement values of the first or the second measurement using the correction term (e.g. by adding the respective measurement values to the correction term).

The first estimated value can be corrected further in order to take into account the statistical deviations of the first measurement from the second measurement. This can be done, for example, by a combination of the optionally corrected measurement values from the first and the second measurement, as will be described in detail below.

The combination of the measurement values from the first and the second measurement and the correction or adaptation of the measurement values from the first and the second measurement can also take place in the reverse order.

According to a preferred example, the first measurement of the vision disorder of the eye is an objective refraction and/or the second measurement of the vision disorder of the eye is a subjective refraction. The measurement values accordingly comprise values of at least one component or refraction component. This at least one component of the measurement values can be a component of a wavefront representation of the vision disorder, its linear combination or variables derived therefrom. The at least one component can e.g. be:
the component of a polar representation (sphere, cylinder and axis),
the component of a curvature matrix representation,
the component of a power vector representation (M, J0 and J45),
the component of a Harris vector representation,
the component of a Zernike polynomial decomposition (Zernike coefficient), or
the component of another suitable characterization of the vision disorder of a spectacle wearer.

The method can furthermore comprise providing data about the measurement accuracies or measurement deviations of the first and the second measurement of the vision disorder. The data can be stored in electronic form (e.g. stored in a database) or in a form (e.g. on paper). The data can be present in tabular form (e.g. as a “look-up table” (LUT)) or be predetermined as a mathematical model, e.g. as a parametric function with identified parameters.

The method can comprise determining the measurement inaccuracies or measurement deviations of the first and the second measurement using statistical analysis, such as a statistical analysis of the data or measurement values (reference measurement values) contained in a data set (reference data set) from previous measurements (e.g. previous first and second measurements or measurements with measuring devices of the first and second type) of different spectacle wearers. The data set (reference data set) can also comprise other measurements, on the basis of which the measurement inaccuracies or measurement deviations of the first and second measurements are determined.

The raw measurement values can be filtered prior to analysis, e.g. on the basis of the following criteria:
the amount of a difference between an addition and a reciprocal object distance in (subjective) near refraction measurement (with positive sign convention) is equal to or less than a predetermined threshold value, optionally equal to or less than 0 dtp, 0.25 dpt or 0.5 dpt;
the visual acuity of the respective spectacle wearer (whose refraction values are contained in the data set) is equal to or greater than a predetermined threshold value, optionally equal to or greater than 1.25 or 1.5 or 1.6;
the resolution of the refraction lenses used for the subjective refraction of a spectacle wearer is equal to or higher than a predetermined threshold value, optionally equal to or higher than 0.5 dpt or 0.25 dpt or 0.125 dpt.

Other criteria are also possible, such as the density of the data in a specific measurement interval.

The determination of the measurement inaccuracies or the measurement deviations of the first and the second measurement can e.g. comprise the following steps: setting a model for the measurement values of the second measurement as a sum of a predicted measurement value and a random variable, wherein the predicted measurement value is modeled as a parametric function of the measurement value of the first measurement and optionally a part of the measurement value of the second measurement;

specifying the parameters of the parametric function by adapting the model to the reference measurements contained in the data set while maximizing the probability distribution of the random variables in the parameter space of the model;

determining a systematic deviation of the first measurement from the second measurement on the basis of the predicted measurement.

The model can be described e.g. by the following equation or the following equation system:

\tilde{P}₂=P_{pred}[\tilde{P}₁, . . . ]+ε,

where:
\tilde{P}₁ denotes the measurement value of the first measurement (in vector form);
\tilde{P}₂ denotes the measurement value of the second measurement (in vector form);
P_{pred} denotes the predicted measurement value (in vector form); and
ε denotes the random variable (in vector form).

The above equation or the above equation system is to be considered separately for each measurement value in the reference data set, i.e. applies to each measurement “i”. Thus, it holds for the i^{th} measurement:

\tilde{P}ⁱ₂=P_{pred}[\tilde{P}ⁱ₁, . . . ]+εⁱ,

Each measurement can therefore be assigned a random variable εⁱ (which can be a vector quantity). All random variables εⁱ come from the same distribution or relate to the same distribution.

If the parametric function is optionally a function of the measurement value of the first measurement and a part of the measurement value of the second measurement, the component of the second measurement that is modeled is preferably not taken into account in the parametric function. Otherwise there is a trivial solution, namely that the random variable is always 0, and the parametric function is identical to the component to be modeled in the second measurement.

The predicted measurement can be modeled by an arbitrary parametric function, e.g. by a polynomial function. For example, the predicted measurement can be a predicted refraction, which can be modeled by one of the following parametric functions:

$$\begin{matrix}{M}_{pred} ( {\tilde{M}}_{obj} , {obj}_{} , {obj}_{} ) = {\sum }_{i = 0}^{4} {a}_{M , i}^{M} {\tilde{M}}_{obj}^{i} + {a}_{J0 , 1}^{M} {obj}_{} + {a}_{J 45 , 1}^{M} {obj}_{} & Model 1 \\ J {0}_{pred} ( {\tilde{M}}_{obj} , {obj}_{} , {obj}_{} ) = {a}_{M , i}^{J 0} {\tilde{M}}_{obj} + {\sum }_{i = 0}^{4} {a}_{J 0 , i}^{J 0} {obj}_{i}^{} + {a}_{J 45 , 1}^{J 0} {obj}_{} & \\ J {45}_{pred} ( {\tilde{M}}_{obj} , {obj}_{} , {obj}_{} ) = {a}_{M , 1}^{J 45} {\tilde{M}}_{obj} + {a}_{J 0 , 1}^{J 45} {obj}_{} + {\sum }_{i = 0}^{4} {a}_{J 45 , i}^{J 45} {obj}_{i}^{} & \\ or & \\ {M}_{pred} ( {\tilde{M}}_{obj} , \overline{J} {0}_{obj} , {obj}_{} , \overline{J} {0}_{sub} , {sub}_{} ) = {\sum }_{i = 0}^{4} {a}_{M , t}^{M} {\tilde{M}}_{obj}^{i} + {a}_{j 0 , 1}^{M} {obj}_{} + {a}_{j 4 b , 1}^{M} {obj}_{} + {b}_{j 0 , 1}^{M} {sub}_{} + {b}_{j 4 b , 1}^{M} {sub}_{} & Model 2 \\ J {0}_{pred} ( {\tilde{M}}_{obj} , {obj}_{} , {obj}_{} , {sub}_{} , {sub}_{} ) = {a}_{M , 1}^{J 0} {\tilde{M}}_{obj} + {\sum }_{i = 0}^{4} {a}_{{I}^{0 , i}}^{J 0} {obj}_{i}^{} + {a}_{j 45 , 1}^{J 0} {obj}_{} + {b}_{M , 1}^{J 0} {\tilde{M}}_{sub} + {b}_{J 45 , 1}^{J 0} {sub}_{} & \\ J {45}_{pred} ( {\tilde{M}}_{obj} , {obj}_{} , {obj}_{} , {sub}_{} , {sub}_{} ) = {a}_{M , 1}^{J 45} {\tilde{M}}_{obj} + {a}_{J 0 , 1}^{J 45} {obj}_{} + {\sum }_{i = 0}^{4} {a}_{J 45 , i}^{J 45} {obj}_{i}^{} + {b}_{M , 1}^{J 45} {\tilde{M}}_{sub} + {b}_{J 0 , 1}^{J 45} {sub}_{} & \end{matrix}$$

where:
(M_{zred}, J0_{pred}, J45_{pred}) denotes the power vector of the predicted refraction;
(\tilde{M}_{obj}, [symbol] _{obj}, [symbol] _{obj}), denotes the power vector of the measurement values from the objective refraction;
(\tilde{M}_{sub}, [symbol] _{sub}, [symbol] _{sub}) denotes the power vector of the measurement values from the subjective refraction;
a_{X,i}^{Y} denote the parameters of the respective parametric function,
Y stands for a power vector component of the power vector of the predicted refraction;
X stands for a power vector component of the power vector of the measured objective refraction.

The specified parameters a_{X,i}^{Y} can be stored in a suitable form (for example as LUT) and be taken into account in the calculation of the estimated value for the vision disorder.

The systematic deviation of the first measurement from the second measurement and corresponding correction terms can be determined on the basis of the predicted measurement according to the model and the subjective and objective measurement values provided. Here, the objective measurement value, the subjective measurement value, or both measurement values can be corrected (for example by adding the respective measurement value to the specific correction term).

It is also proposed to minimize the statistical measurement errors or measurement inaccuracies by combining the measurement values from the first measurement and the second measurement (for example the subjective and the objective refraction values). It has proven particularly advantageous to calculate the estimated value of the vision disorder of the eye by forming a weighted average of the measurement values from the first and second measurements, the first measurement or the components of the measurement value from the first measurement being weighted with first weights and the second measurement or the components of the measurement value from the second measurement being weighted with second weights, and the sum of the first and the second weight for the respective component is equal to 1. Since the measurement values are vector quantities in principle (i.e. variables with several components), the individual components (e.g. power vector components) are generally weighted with different weights. If the respective measurement value has only one component (e.g. the spherical equivalent), the component from the first measurement is weighted with a first weight and the component from the second measurement is weighted with a second weight.

Preferably, among the first measurement and the second measurement, the measurement with the lower measurement inaccuracy is weighted with higher weights. Preferably, the measurement values from the first measurement and/or the measurement values from the second measurement are corrected or modified beforehand in order to reduce the statistical deviations between the first and the second measurement.

The weights are preferably dependent on the measurement values of the vision disorder. The measurement values can e.g. comprise an addition and/or a spherical equivalent and the weights can be dependent on the addition and/or the difference between the measurement value of the spherical equivalent from the first measurement and the measurement value of the spherical equivalent from the second measurement. According to one aspect, a novel weighting is proposed in order to minimize the statistical measurement inaccuracies or deviations of an objective and a subjective measurement of the vision disorder.

If, for example, the addition is equal to or higher than a predetermined value (e.g. 1.75 dpt or 2.0 dpt or 2.25 dpt or 2.5 dpt) or equivalently the accommodation ability is equal to or lower than a predetermined value (e.g. 0.75 dpt or 0.5 dpt or 0.25 dpt or 0 dpt), and if the difference or the disparity ΔM between the objective spherical equivalent and the subjective spherical equivalent is not great (e.g. in the interval −0.75 dpt<ΔM<+0.75 dpt or −0.5 dpt<ΔM<+0.5 dpt), the weight of the subjective spherical equivalent is between 0.3 and 0.7.

If the addition is equal to or higher than a predetermined value (e.g. 1.75 dpt or 2.0 dpt or 2.25 dpt or 2.5 dpt) or equivalently the accommodation ability is equal to or lower than a predetermined value (e.g. 0, 75 dpt or 0.5 dpt or 0.25 dpt or 0 dpt), and if the amount of the difference between the objective spherical equivalent and the subjective spherical equivalent is large (e.g. greater than 1.5 dpt or 1.0 dpt or 0.5 dpt), the weight of the subjective spherical equivalent is greater than or equal to 0.8 or 0.9 or 0.95 or 0.99. The value can even be 1.

If the addition is equal to or less than a predetermined value (e.g. equal to or less than 1.5 dpt or 1.25 dpt or 1.0 dpt or 0.75 dpt or 0.5 dpt) or equivalently the accommodation ability is equal to or greater than a predetermined value (e.g. equal to or higher than 1.0 dpt or 1.25 dpt or 1.5 dpt or 1.75 dpt or 2.0 dpt), and if the difference ΔM between the objective spherical equivalent and the subjective spherical equivalent is negative and smaller than a predetermined value, (e.g. smaller than −0.5 dpt or −1.0 dpt or −1.5 dpt), the weight of the objective spherical equivalent is small, e.g. 0.5 or 0, 4 or 0.3 or 0.2 or 0, 1 or 0.05 or 0.01. The value can even be 0.

If the addition is lower than a predetermined value and the difference ΔM between the objective spherical equivalent and the subjective spherical equivalent is not great (e.g. in the interval −0.75 dpt<ΔM<+0.75 dpt or −0.5 dpt<ΔM<+0.5 dpt), the subjective and objective spherical equivalents are weighted similarly as in the case of the presence of (relatively) high presbyopia. The weight of the subjective spherical equivalent can e.g. be between 0.3 and 0.7 or between 0.4 and 0.6.

The weights can further also depend on other components of the refraction (refraction components), such as the components J0 and J45 in the power vector representation.

The measurements of the vision disorder can also comprise at least one astigmatic component (for example the power vector components J0 and J45), wherein the subjective and objective astigmatic components can be weighted with constant weights. For example, the weight for the subjective astigmatic component can be 0.7 and the weight for the corresponding objective astigmatic component 0.3. Other values are also possible and can express that both measurements have the same statistical inaccuracy (both weights 0.5) or that the subjectively determined astigmatic components have a lower statistical inaccuracy (e.g. weight objective: 0.7, weight subjective: 0.3).

Preferably, the measurement inaccuracies or measurement deviations of the first and the second measurement are preferably determined or quantified for the same object distance, such as object distance Infinite. Furthermore, the measurement inaccuracies or measurement deviations are preferably determined or quantified at a distance to the eye that is identical for all data. The method can accordingly comprise converting raw objective and/or subjective refraction values to a common distance to the eye or to a common plane or surface, wherein the distance may be the distance to the corneal vertex or the entrance pupil of the eye.

Further preferably, the measurement inaccuracies or the measurement deviations of the first and the second measurement are determined or quantified separately for different apparatuses for determining objective refraction values.

The above method can be carried out using an appropriately designed device. The device can comprise a computing or data processing device (in particular a computer or computer system), which is programmed to carry out the method and in particular to calculate the estimated value. Furthermore, the device can have suitable interfaces that enable the transmission or input or readout of measurement values from a first and a second measurement. The device can also comprise a storage unit that stores the measurement values from the first and the second measurement and, if appropriate, previously determined measurement inaccuracies or measurement deviations (for example in tabular form or in the form of a model).

The device for determining the vision disorder of an eye of a spectacle wearer can further comprise at least one measuring device of a first type for performing the first measurement, in particular a measuring device for performing an objective refraction measurement. Preferably, the computing device is designed, as described above, to at least partially compensate for the systematic deviations of the measurement values obtained with the measuring device for performing an objective refraction measurement (objective measuring device) from the measurement values obtained with a subjective measurement.

The device can further comprise a second measuring device of a second type for performing the second measurement, in particular a measuring device for performing a subjective refraction measurement.

The method for determining the vision disorder of a spectacle wearer can be part of a method for ordering and/or producing a spectacle lens. Accordingly, the device for determining the vision disorder of a spectacle wearer can be part of a device for ordering and/or producing a spectacle lens. The method for ordering and/or producing a spectacle lens can further comprise setting a target power of the spectacle lens on the basis of the determined vision disorder. The target power of the spectacle lens is stipulated such that the determined vision disorder is corrected at least partially, preferably substantially, in at least one reference point of the spectacle lens (such as in the distance reference point or in the prism reference point or in the centration cross and optionally in the near reference point). The method can furthermore comprise calculating and manufacturing the spectacle lens, the spectacle lens being calculated and manufactured such that its power in the at least one reference point is substantially equal to the target power. Preferably, the calculation is carried out in a wearing position individually predetermined for the spectacle wearer or in an average wearing position. The wearing position can be characterized by parameters such as corneal vertex distance, ocular center of rotation distance, forward inclination, face form angle, pupillary distance, pupil diameter, etc.

A further aspect of the invention relates to a spectacle lens or a set of a spectacle lens and a specification of the vision disorder the spectacle lens is to correct, wherein the spectacle lens can be produced according to the above method. A series of spectacle lenses or sets of spectacle lenses with associated specifications of the vision disorder is also proposed. Thus, the specification of the vision disorder to be corrected by a specific spectacle lens can be considered to be part of the spectacle lens.

The spectacle lens has a first power P_A in a reference point of the spectacle lens. The reference point can e.g. be the distance reference point, the prism reference point, the centration cross, the near reference point or another suitable reference point. As described above, the power can have several components, such as a spherical and/or an astigmatic component.

The vision disorder (which can be part of the specification for the spectacle lens) can be characterized by a first measurement value P_A1 and a second measurement value P_A2, wherein the measurement values can comprise several components (such as a spherical, an astigmatic component, etc.). The components of the measurement values of the vision disorder generally correspond to the components of the power in the reference point of the spectacle lens.

The first measurement value P_A1 and the second measurement value P_A2 are obtained using different measurements. In particular, the first measurement value P_A1 is obtained using a measuring device of the first type for measuring the vision disorder and the second measurement value P_A2 is obtained using a measuring device of the second type for measuring the vision disorder. As a rule, the first measurement value P_A1 and the second measurement value P_A2 differ in at least one component X.

The component X of the power P_A present in the reference point of the spectacle lens is closer to the component X of the measurement value among the measurement values P_A1 or P_A2 that is obtained from the measuring device with the lower inaccuracy in the measurement of the component X. As explained above, the components of the measurement values P_A1 and P_A2 can be components of a wavefront representation of the vision disorder, their its combination or variables derived therefrom. Preferably, the component X of the power P_A present in the reference point of the first spectacle lens and the component X of the first measurement value of the first eye P_A1 are substantially identical.

The spectacle lens can be a single vision lens (with or without astigmatic power) or a progressive lens.

The above spectacle lenses can form a series of spectacle lenses with different powers in the at least one reference point, wherein the spectacle lenses correct different vision disorders. Such a series can comprise e.g. at least three spectacle lenses with different powers in the reference point:
a first spectacle lens A for correcting a first vision disorder,
a second spectacle lens B for correcting a second vision disorder; and
a third spectacle lens C for correcting a third vision disorder.

The first, second and third vision disorders can each be characterized by two different measurement values, the two measurement values being obtained using different measuring devices for measuring the vision disorder. The measuring device or measuring devices of the first type (first measuring device(s)) can be a measuring device or measuring devices for measuring the subjective refraction. The measuring device or measuring devices of the second type (second measuring device(s)) can be a measuring device or measuring devices for measuring the objective refraction.

Each measurement value can comprise several components (e.g. a spherical, an astigmatic component, etc.). The measurement values obtained with the first measuring device(s) differ from the measurement values obtained with the second measuring device(s) in at least one component.

In the at least one reference point, the spectacle lens A has a first power P_A, the spectacle lens B a second power P_B, and the spectacle lens C a third power P_C. The first vision disorder is characterized by a first measurement value P_A1 and a second measurement value P_A2. The second vision disorder is characterized by a first measurement value P_B1 and a second measurement value P_B2. The third vision disorder is characterized by a first measurement value P_C1 and a second measurement value P_C2.

The first reference point can be the distance reference point, the prism reference point, the centration point or the centration cross, the near reference point or another suitable reference point. The first reference point can be marked or labeled in or on the spectacle lens using a permanent or non-permanent marking.

The first measurement values P_A1, P_B1 and P_C1 determined with the measuring device(s) of the first type are identical in terms of components. The components X of the second measurement values P_A2, P_B2 and P_C2 determined with the measuring device(s) of the second type all differ pairwise. The component X of the first power P_A and the component X of the first measurement value P_A1 are almost identical. For the components X of the power of the i^{th} spectacle lens present in the reference point, X_i, where i=A, B or C, and for the components X of the second measurement values of the i^{th} eyes, X_i2, the following relationships preferably apply:

(X_B−X_A)/(X_B2−X_A2) unequal (X_C−X_A)/(X_C2−X_A2);

abs(X_B2−X_A2)<abs(X_C2−X_A2); and

signum(X_B2−X_A2)=signum(X_C2−X_A2),

where the function abs(x) specifies the absolute value of the argument x and the function signum (x) is the sign function that assigns the sign to the argument x.

The lenses can be single vision lenses (Add=0 dpt) or progressive lenses (multifocal lenses) (Add #0 dpt), wherein all progressive lenses in the series have the same additions.

Preferably, for single vision lenses and progressive lenses having the same addition Add with an addition Add<=1.5, optionally 1.25 dpt, the following relationships apply to components X of the power of the i^{th} spectacle lens present in the reference point, X_i, and for the components X of the second measurement values of the i^{th} eyes, X_i2:

(X_B−X_A)/(X_B2−X_A2)<(X_C−X_A)/(X_C2−X_A2) falls X_B2−X_A2>0,

X_C2−X_A2>0,

and

(X_B−X_A)/(X_B2−X_A2)>(X_C−X_A)/(X_C2−X_A2) falls X_B2−X_A2<0,

X_C2−X_A2<0.

Preferably, for progressive lenses with an addition Add>=2 dpt, the following relationships apply for the components X of the power of the i^{th} lens present in the reference point, X_i, and for the components X of the second measurement values of the i^{th} eyes, X_i2:

(X_B−X_A)/(X_B2−X_A2)>(X_C−X_A)/(X_C2−X_A2) falls X_B2−X_A2>0,

X_C2−X_A2>0,

and

(X_B−X_A)/(X_B2−X_A2)>(X_C−X_A)/(X_C2−X_A2) falls X_B2−X_A2<0,X_C2−X_A2<0.

The component X can be the spherical equivalent, for example.

The series of spectacle lenses can comprise a fourth spectacle lens D for correcting a fourth vision disorder and a fifth spectacle lens E for correcting a fifth vision disorder. The spectacle lens D has a fourth power P_D in the reference point. The spectacle lens E has a fifth power P_E in the reference point. The fourth vision disorder is characterized by at least a first measurement value P_D1 and a second measurement value P_D2.

The fifth vision disorder is characterized by at least a first measurement value P_E1 and a second measurement value P_E2.

The measurement values P_D1 and P_E1 are obtained using the measuring device(s) of the first type for measuring the vision disorder. The measurement values P_D2 and P_E2 are obtained using the measuring device(s) of the second type for measuring the vision disorder.

The measurement values P_D1, P_D2, P_E1 and P_E2 each preferably consist of several components. The first measurement value P_D1 and the second measurement value P_D2 can differ in at least one component X. The first measurement value P_E1 and the second measurement value P_E2 can also differ in at least one component X.

Furthermore, the following conditions are preferably satisfied:
the values P_A1, P_D1 and P_E1 are identical in terms of components:
the components X of the second measurement values P_A2, P_D2 and P_E2 of the first, fourth and fifth eyes determined with the measuring devices of the second type all differ pairwise,
the component X of the first power P_A present in the reference point of the first spectacle lens and the component X of the first measurement value of the first eye, P_A1, are almost identical, and
for the components X of the power of the i^{th} spectacle lens present in the reference point, X_i, and for the components X of the second measurement values of the i^{th} eyes, X_i2, the following relationships apply:

X_D2−X_A2>0,

X_E2−X_A2<0,

X_D−X_A>0 and

X_E−X_A<0.

The series can also comprise other lenses with different powers for correcting different vision disorders.

### BRIEF DESCRIPTION OF THE DRAWINGS

Preferred embodiments of the present invention will be described below by way of example with reference to accompanying figures. Individual elements of the embodiments described are not limited to the respective embodiment. Instead, elements of the embodiments can be combined with one another as required and new embodiments can be created thereby. The figures show:

FIG. 1 the systematic deviations of objective and subjective wavefronts for two different aberrometers (model 1);

FIG. 2 the systematic deviations of objective and subjective wavefronts for two different aberrometers (model 2);

FIG. 3 the weights of the subjective spherical equivalent g_{sub} according to a first example;

FIG. 4 the weights of the subjective spherical equivalent g_{sub} according to a third second example (FIG. 4A) and a third example (FIG. 4B);

FIG. 5 the change in the estimated value of the vision disorder obtained using two different methods for a first aberrometer as the difference of the values obtained using the different methods;

FIG. 6 the change in the estimated value of the vision disorder obtained using two different methods for a second aberrometer as the difference of the values obtained using the two different methods;

FIGS. 7 to 10 exemplary spectacle lenses;

FIGS. 11 to 19 the difference between an estimated value of the spherical equivalent and a measured subjective spherical equivalent as a function of the difference between a measured objective spherical equivalent and a measured subjective spherical equivalent for different additions.

### DETAILED DESCRIPTION

In the context of the present application, reference is made to the following technical terminology:

The measurement of the vision disorder of an eye comprises, in particular, a subjective refraction determination, an objective refraction determination (e.g. with a refractometer or an auto refractometer) or a wavefront measurement. The objective refraction determination or the wavefront measurement are examples of an objective refraction.

A wavefront representation is understood to mean a parameterization of a 2-dimensional wavefront in 3-dimensional space. This includes, in particular, the following parameterizations:
polar representation (with the components sphere, cylinder and axis), curvature matrix representation, power vector representation (with the components M, J0 and J45),
Harris vector representation, Zernike polynomial decomposition (here, components are the Zernike coefficients).

An objective refraction is understood to mean a determination or the measurement values of the vision disorder of an eye obtained by the determination, wherein the person measured with a measuring device used during the objective refraction does not have to assess the quality of vision of the image being viewed. Objective refractions or objective measurement values can be measured using wavefront scanners or auto refractometers, for example.

A subjective refraction is understood to mean the determination or the measurement values of the vision disorder of an eye, wherein the person refracted has to assess the quality of vision of the image being viewed or has to solve a visual task, e.g. recognition of optotypes, and has to communicate the solution. Subjective refractions can e.g. be established by experts with the help of refraction spectacles, into which refraction lenses are introduced, or with the help of phoropters. A subjective refraction can also comprise a subjectively determined near addition, the so-called addition.

The reference point is the visual point of a spectacle lens, in which the power of the spectacle lens is predetermined by the position and orientation of the spectacle lens in front of the eye and by the vision disorder of the eye for which the spectacle lens is to be used. This can be the distance reference point, the prism reference point, the centration point, the centration cross, the near reference point, etc. With regard to the definition of the reference point, reference is made to the standards DIN EN ISO 21987 (in particular points 3.5 to 3.11) and DIN EN ISO 13666 (in particular points 5.12 to 5.17).

With regard to the technical terminology used, reference is made in particular to WO 2009/007136 A1, the publication by L. Thibos et al., Journal of Vision April 2004, Vol. 4, 9. doi: 10.1167/4.4.9 and the publication Iskander et al., Ophthal. Physiol. Opt. 2007 27: 245-255, the corresponding explanations of which represent an integral part of the disclosure of the present application.

A first example relates to a method for determining the vision disorder of an eye of a spectacle wearer, comprising:
providing measurement values from a first and a second measurement of the vision disorder of the eye of the spectacle wearer;
calculating an estimator or estimated value for the vision disorder of the eye of the spectacle wearer based on the measurement values from the first and the second measurement, wherein measurement inaccuracies or measurement deviations of the first and the second measurements of the vision disorder are taken into account in the calculation of the estimated value of the vision disorder.

If several measurements of the vision disorder of an eye are known, they can, according to an example of the invention and depending on their measurement inaccuracies, be used to calculate an estimator of the vision disorder. Preferably, the estimator is closer to the measurement that has the lower measurement inaccuracy.

Basically, two types of the measurement inaccuracy can be distinguished: It is known that there are systematic deviations that do not change when a measurement is repeated. It is also known that there are so-called statistical or random deviations in the measurement value, which, when a measurement is repeated, can assume different values and cannot be predicted.

One possibility of calculating the estimator or the estimated value of the vision disorder is therefore to take into account the systematic deviations of the measurements in the determination of the estimator or the estimated value of the vision disorder. In this case, the measurement value afflicted with the systematic deviation can be corrected by this systematic deviation toward the other measurement. If the estimator of the vision disorder is then calculated from the corrected measurement value of the measurement afflicted with systematic errors and the measurement value of the measurement not afflicted with systematic errors, e.g. with the aid of an average, the estimator is closer to the measurement value not afflicted with systematic errors.

Another possibility for calculating the estimator of the vision disorder is to take into account the statistical deviations of the measurements in the determination of the vision disorder estimator. This can preferably be done with the aid of a weighted average. Here, the weights are preferably chosen such that the less imprecise measurement is given the higher weight. In the case of normally distributed variables, the weights can be selected proportionally to the reciprocal variance of the measured variable. In cases where there is no normal distribution, a choice of weights based on experience may be necessary. The less precise measurement can e.g. be assigned weights of 0.3, 0.2, 0.1, 0.05, 0.01 or less, even up to a weight of 0. The more precise measurement can be assigned a weight of 0.7, 0.8, 0.9, 0.95, 0.99 or more, even up to a weight of 1. If both measurements are similarly accurate, they can each obtain a weight of 0.5. The weights can be chosen such that their sum is 1. In this case, dividing by the sum of the weights is no longer necessary when forming the weighted average.

Since also larger statistical deviations can occur in the measurement of vision disorder, e.g. due to accommodation, fluctuations in the accommodation state, lens opacity, visual acuity, but also other variables, it can be advantageous to choose the weights depending on the difference in the measurement values of the vision disorder. For example, for persons who can hardly accommodate and therefore have been prescribed an addition of 1.75 dpt, 2.0 dpt, 2.25 dpt, 2.5 dpt or higher, the subjective and objective measurement values of the spherical equivalent should hardly differ. If there is a slight difference, the spherical equivalents from the subjective and objective measurements can be added in a weighted manner, with possible weights of the subjective and objective spherical equivalents between 0.3 and 0.7 making sense. If there are major differences, however, the subjective refraction is more likely to be trusted, since the person did already get an idea of the quality of vision through such a lens during the subjective refraction. In this case, higher weights (e.g. 0.8, 0.9, 0.95, 0.99, or higher, or even 1) should be selected for the subjective refraction.

For presbyopic persons who can still accommodate quite a lot, i.e, persons who have been prescribed an addition of 1.5 dpt, 1.25 dpt, 1.0 dpt, 0.75 dpt, 0.5 dpt or lower, or else people who are not presbyopic, i.e. effectively have an addition of 0, device myopia can increasingly occur, for example. In this case, for a more myopic spherical equivalent of the objective refraction compared to the spherical equivalent of the subjective refraction, the former is to be weighted less, e.g. with weights of 0.3, 0.2, 0, 1, 0.05, 0.01 or less up to a weight of 0. However, if the spherical equivalents of the subjective refraction are similar, then it is advisable to choose a weighting as with presbyopes with high additions. If the subjective spherical equivalent is more myopic than the objective spherical equivalent, e.g. by 0.5 dpt, the person could have accommodated during the subjective refraction. Typically, a lower weight would have to be selected for the subjective spherical equivalent, but since device myopia can often occur in the objective refraction, the weight of the subjective spherical equivalent can also be selected to be somewhat higher, e.g. between 0.4 and 0.6.

In practice, both systematic and statistical deviations from measurement values of vision disorder occur. In this case, the systematic deviations are preferably corrected first, and then the corrected measurement values are combined in a weighted manner on the basis of the statistical measurement uncertainty.

Also in this case is the estimator or estimated value of the vision disorder calculated in this way closer to the measurement value having the lower measurement inaccuracy.

An exemplary method for determining the vision disorder of a spectacle wearer comprises the following steps:
1) matching of the subjective and/or objective refractions in order to eliminate systematic differences in the two measurement methods;
2) combination of the refractions thus matched to one another by forming a weighted average.

Step 1—Matching of the Subjective and Objective Refractions

Quantifying the Systematic Differences Between Subjective and Objective Refraction

In order to match the subjective and objective refraction to one another, or to compensate for the systematic differences between subjective and objective refraction, these are first quantified. To this end, a sufficiently large data set must first be available and processed as described below.

The systematic differences between subjective and objective refraction are preferably quantified for the object distance Infinite, i.e. for the so-called distance prescription, since it is much more accurate in terms of the spherical equivalent than the near refraction.

Typically, the systematic differences are also different for different apparatus models (for example aberrometer models) from different manufacturers. It is therefore advantageous to take the information about the apparatus model into account when acquiring the data and to determine the systematic differences separately for each aberrometer model.

In order to calculate an objective refraction from the wavefront measured with an apparatus (such as an aberrometer, a wavefront scanner, etc.), the second-order wavefront is preferably determined using a so-called metric. Possible metrics are described e.g. in L. Thibos et al., Journal of Vision April 2004, Vol. 4, 9. doi: 10.1167/4.4.9. However, other metrics are also readily conceivable and known to a skilled person.

The objective refraction data can be calculated e.g. with the help of the refractive RMS metric by Iskander et al. (Iskander et al., Ophthal. Physiol. Opt. 2007 27:245-255) from the wavefronts up to the 7th radial order after the Zernike wavefront has been scaled centrally to the photopic pupil, which was measured in the topography measurement of the apparatuses used (such as aberrometers).

It is advantageous to know the pupil diameter at which the subjective refraction was carried out, wherein this can be done by direct measurement, estimation from other measurement parameters or estimation based on experience. If the pupil diameter is known, the objectively measured wavefront can first be scaled to this pupil diameter, and then the power vector associated with the pupil can be calculated. It is also advantageous to take the position of the pupil into account in the scaling of the wavefront, if it differs in the wavefront measurement and the subjective refraction.

If the pupil diameter is unknown in the subjective refraction, it can be determined as an estimated value e.g. from the illuminance falling on the eye during the subjective refraction and—if available—also other variables such as the largest (with weak lighting) and smallest (with strong lighting) determined pupil diameter of the refracted person.

The subjective and objective refraction are preferably compared at a distance to the eye that is identical for all data. This distance can be arbitrary. However, it has proven advantageous to first convert the subjective refraction to the distance to the eye at which the wavefronts measured by the aberrometer, wavefront scanner, etc. are located as well. This avoids the complex propagation of the objective wavefront often containing higher order aberrations. Possible sensible distances to the eye are, for example, the corneal vertex or the entrance pupil. The data shown in the figures are given for wavefronts or refractions at the corneal vertext.

In principle, however, it is also possible to convert the objective refraction to a different distance to the eye, wherein the wavefront containing the higher order aberration(s) also has to be correctly propagated.

In order to be able to analyze as much data as possible with a uniform model, both subjective and objective refractions of the left eye can be mirrored vertically. If power vectors are used, the sign of the J45 power vector component must be reversed. Since the distributions of the higher order aberrations in the left and right eyes are mirror symmetrical, the refractions of the right and mirrored left eyes can be analyzed together in this way.

However, it can also be advantageous not to carry out this mirroring, e.g. if the aberrometer would not correctly measure a mirrored wavefront. In this case, the corrections for the left and right eyes must be evaluated and carried out separately.

In order to quantify the systematic differences between subjective and objective refraction, it is also advantageous to evaluate only that part of the data sets that has no or only a few artifacts. Possible artifacts are, for example, device myopia or age-related eye diseases. For example, only that part of the data can be used in which the prescribed addition differs only slightly from the reciprocal object distance in the near refraction, i.e. the amount of the difference of addition and the reciprocal object distance (with a positive sign convention) must not be greater than a predetermined threshold value. The threshold value can e.g. be 0 dpt, 0.25 dpt or 0.5 dpt. This reduces the number and extent of device myopia in the data set.

Another limitation is that of high visual acuity, which avoids refraction artifacts from amblyopic persons or other anomalies of central vision. In this way, only that part of the data can be used for which the visual acuity is higher than a predetermined threshold value. Possible limits here are e.g. 1, 25 or 1.6 or more with a decimal visual acuity monocular. This condition is preferably satisfied in both eyes.

It is also advantageous to only use data from refractors who use refraction lenses with a sufficiently high resolution for subjective refraction (e.g. 0.25 dtp or preferably 0.125 dpt for spherical refraction lenses, and 0.5 dpt or even better 0.25 dpt for cylindrical lenses). This can be determined from the distribution of the lens orders of the respective refractor.

To adapt the data, a model of the subjective refraction is preferably set up first, with which a predicted refraction can be calculated from the measured objective refraction and possibly other measured variables, and with which deviations of the actually measured subjective refraction from the predicted objective refraction can be statistically quantified. This model is adapted to the data in a subsequent step.

It is advantageous to adapt the model to the subjective refraction in the power vector space (see L. Thibos et al.: Power Vectors: An Application of Fourier Analysis to the Description and Statistical Analysis of Refractive Error, Optometry and Vision Science 74, 6, 367-375), and to specify the measured and systematically differing subjective and objective refractions as power vectors of the subjective refraction \tilde{P}_{sub}=(\tilde{M}_{sub}, [symbol] _{sub}, [symbol] _{sub}) and the objective refraction \tilde{P}_{obj}=(\tilde{M}_{obj}, [symbol] _{obj}, [symbol] _{obj}). Here, the tilde in the notation refers to the uncorrected (raw) data in the data set.

Within the framework of the model, the actually measured subjective refraction is described based on the predicted refraction, P_{pred}=(M_{pred}, J0_{pred}, J45_{pred}), and the random variables ε_{M}, ε_{J0} and ε_{J45}, the latter modeling both the measurement inaccuracy of the apparatus and that of the refracting person:

\tilde{M}_{sub}=M_{pred}+ε_{M}

[symbol]

_{sub}=J0_{pred}+ε_{J0}

[symbol]

_{sub}=J45_{pred}+ε_{J45}. (1)

The following is an abbreviated form for this system of equations

\tilde{P}_{sub}=P_{pred}└\tilde{P}_{obj}, . . . ┘+ε (1a)

where E is the power vector Equation 1 a applies to every measurement of the data set, so that for the i^{th} measurement

\tilde{P}ⁱ_{sub}=P_{pred}[ [symbol] _{obj}, . . . ]+εⁱ (1b)

can be written.

The power vector of the predicted refraction P_{pred} depends on the objective refraction. If necessary, it can also depend on additional variables such as the subjective refraction, or e.g. on pupil diameters, or other measurement variables arising during a refraction or an objective measurement (e.g. an aberrometer measurement).

One criterion by which the model can be adapted to the data is the maximization of the probability (density) of the random variables ε_{M}, ε_{J0} and ε_{J45} in the parameter space of the model with the data set used, which will also be referred to as “fit” in the following. Suitable methods are, for example, “maximum likelihood” methods, which maximize the probability of generating the data set to be adapted, the so-called likelihood. In the models disclosed here, the likelihood is given by the following equation

prob({ε_{X}};Parameter)=Πᵢprob(ε_{X}ⁱ;Parameter), (2)

where prob({ε_{X}};Parameter) is the probability density of the entire data set with given parameters of the model, and prob(ε_{X}ⁱ;Parameter) is the probability density of an individual measurement from the data set.

As a possible alternative to the maximum likelihood method, the least squares method can be used, which can also be considered equivalent to the “maximum likelihood” method with a normally distributed likelihood. Previous knowledge about the parameters used can also be taken into account in the models, which is possible according to Bayesian data analysis.

The random variable ε_{X} of the measurement inaccuracy of the power vector component X (where X stands for M, J₀ or J₄₅) can be described e.g. by superimposing a uniform distribution, also known as a equal distribution (e.g. in the range of −20 to +20 dpt or in another suitable power range) and a Voigt distribution with the Gaussian width σ^{X} (as standard deviation) and the Lorentz width γ^{X} (as full width at half maximum). The uniform distribution can describe large “outliers” in the data, which occur with the probability p₀^{X}. The Voigt distribution, which occurs with a probability of 1−p_{c}^{X}, describes a successful measurement which, however, can also generate moderate outliers. Overall, the random variable ε_{X} can be distributed as follows:

prob(ε_{X}ⁱ;Parameter)=prob(ε_{X}ⁱ;pₒ^{X},σ^{X},γ^{X})=pₒ^{X}unif(ε_{X}ⁱ;min=−20,max=20)+(1−pₒ^{X})Voight(ε_{X}ⁱ;σ^{X},γ^{X} (3)

As an alternative to the Voigt distribution, the normal distribution can be selected, but with poorer results. The term with the uniform distribution is particularly important, as it is able to intercept large outliers.

For example, only the power vector components of the objective refraction can be used as input variables to calculate the predicted refraction. Here, the calculation can take place using an arbitrary parameterizable function, for example with the aid of polynomials. An exemplary model is the model (Model 1) described by the system of equations (2):

$$\begin{matrix}{M}_{pred} ( {\tilde{M}}_{obj} , {obj}_{} , {obj}_{} ) - {\sum }_{i = 0}^{4} {a}_{M , i}^{M} {\tilde{M}}_{obj}^{i} + {a}_{J 0 , 1}^{M} {obj}_{} + {a}_{J 45 , 1}^{M} {obj}_{} & ( 4 ) \\ J {0}_{pred} ( {\overline{M}}_{obj} , {\overline{J 0}}_{obj} , {obj}_{} ) = {a}_{M , 1}^{I o} {\overline{M}}_{obj} + {\sum }_{i = 0}^{4} {a}_{J 0 , i}^{Jo} {\overline{J 0}}_{obj}^{i} + {a}_{J 45 , 1}^{J 0} {obj}_{} & \\ J {45}_{pred} ( {\tilde{M}}_{obj} , {obj}_{} , {obj}_{} ) = {a}_{M , 1}^{J 45} {\tilde{M}}_{obj} + {a}_{J 0 , 1}^{J 45} {obj}_{} + {\sum }_{i = 0}^{4} {a}_{J 45 , i}^{J 45} {obj}_{i}^{} & \end{matrix}$$

Here, a_{X,i}^{Y} are the model parameters for the fit of the power vector component Y of the subjective refraction, which interacts with the objective power vector components X of the objective refraction. Here, Y stands for M_{pred}, J0_{pred} or J45_{pred} and X for \tilde{M}_{obj}, [symbol] _{obj}, [symbol] _{obj}.

The individual power vector components M_{pred}, J0_{pred} or J45_{pred} of the predicted refraction are preferably functions of all three components of the power vector of the measured (raw) objective refraction.

In an alternative model, the information from the non-fitted components of the subjective power vector is also used to calculate the predicted refraction. The calculation can take place using an arbitrary parameterizable function, for example with the aid of polynomials. An exemplary model is the model (Model 2) described by the system of equations (3):

$$\begin{matrix}{M}_{pred} ( {\tilde{M}}_{obj} , {obj}_{} , {obj}_{} , {sub}_{} , {sub}_{} ) = {\sum }_{i = 0}^{4} {a}_{M , i}^{M} {\tilde{M}}_{obj}^{i} + {a}_{J 0 , 1}^{M} {obj}_{} + {a}_{j 45 , 1}^{M} {obj}_{} + {b}_{J 0 , 1}^{M} {sub}_{} + {b}_{J 45 , 1}^{M} {sub}_{} & ( 5 ) \\ J {0}_{pred} ( {\tilde{M}}_{obj} , {obj}_{} , {obj}_{} , {sub}_{} , {sub}_{} ) = {a}_{M , 1}^{J 0} {\tilde{M}}_{obj} + {\sum }_{i = 0}^{4} {a}_{J 0 , i}^{J 0} {obj}_{i}^{} + {a}_{J 45 , 1}^{j 0} {obj}_{} + {b}_{M , 1}^{J 0} {\tilde{M}}_{sub} + {b}_{J 45 , 1}^{J 0} {sub}_{} & \\ J {45}_{pred} ( {\tilde{M}}_{obj} , {obj}_{} , {obj}_{} , {sub}_{} , {sub}_{} ) = {a}_{M , 1}^{J 45} {\tilde{M}}_{obj} + {a}_{J 0.1}^{J 45} {obj}_{} + {\sum }_{i = 0}^{4} {a}_{J 45 , i}^{J 45} {\tilde{M}}_{sub} + {b}_{J 0 , 1}^{J 45} {sub}_{} & \end{matrix}$$

Due to the possible interaction of subjective and objective power vector components, errors in the subjective refraction determination can also be modeled or taken into account. In this way, it is possible to model errors from refraction practices, such as changing the cylinder while the sphere remains the same, which arise from a lack of resolution in the refraction lenses or the optician's ignorance.

As an alternative to the “maximum likelihood” model described above, estimators of the subjective refraction such as the running median of the power vector of the subjective refraction can also be calculated. A parameterizable description of the predicted power vector, such as using equation system 2 or 3, can then be adapted to the calculated estimator of the subjective refraction using the least squares method. Of course, estimators other than the average value or median can also be used, provided that their errors are approximately distributed normally before the least squares method is used. In contrast, using the pure average value or direct adjustment of the data with the least squares method is not advantageous due to possible outliers in the data.

Examples of the parameter sets belonging to models 1 and 2 are shown in tables 1 and 2. Tables 1 and 2 show the fit results for two different aberrometers (aberrometer 1 and aberrometer 2). The parameters a_{Y,i}^{X} quantify the systematic deviations, the other parameters quantify the measurement uncertainties of the aberrometers. The symbol “*” in tables 1 and 2 means that the corresponding variable is the dependent variable that was to be predicted, so there is no corresponding parameter. In particular, the asterisks refer to the fact that there is no corresponding parameter, otherwise the solution of equation 1 a would be trivially fulfilled with the missing parameter=1 and all others=0).

Table 1 contains the fit results of the model without additional influence of subjective power vector components (model 1). Table 2 contains the fit results of the model with additional influence of subjective power vector components (model 2).

TABLE 1

| | Aberrometer 1 | | | Aberrometer 2 | | |
|---|---|---|---|---|---|---|
| X | M | J0 | J45 | M | J0 | J45 |
| [symbol] | −5.988559e+00 | −14.683359254 | −1.030284e+01 | −8.260018e+00 | −9.102539677 | −1.849467e+01 |
| [symbol] | −1.473851e+00 | −2.260253864 | −2.399429e+00 | −1.484472e+00 | −2.257872473 | −2.412253e+00 |
| [symbol] | −3.708547e+00 | −4.014985072 | −4.173024e+00 | −3.429828e+00 | −3.995043504 | −4.137689e+00 |
| [symbol] | 2.597766e−02 | −0.005823866 | 1.167734e−03 | −8.455234e−02 | −0.002214542 | −1.396330e−03 |
| [symbol] | 9.553421e−01 | 0.003854467 | 3.013793e−04 | 9.448156e−01 | 0.003801166 | 8.891828e−04 |
| [symbol] | −1.089862e−02 | 0.884505067 | −2.097478e−02 | −2.125544e−02 | 0.880071958 | −1.653286e−02 |
| [symbol] | −2.055721e−02 | 0.018582216 | 8.162184e−01 | −1.948651e−02 | 0.012354087 | 8.022532e−01 |
| [symbol] | −7.955925e−03 | 0.033536394 | 8.674891e−03 | −9.785492e−03 | 0.029741058 | 4.053460e−02 |
| [symbol] | −7.773469e−05 | 0.010410193 | 4.275422e−02 | 4.552031e−05 | 0.016098649 | 3.814216e−02 |
| [symbol] | 7.617699e−05 | −0.006240530 | −5.976109e−03 | 1.110717e−04 | −0.003251211 | −1.156952e−02 |

[symbol]

indicates data missing or illegible when filed

TABLE 2

| | Aberrometer 1 | | | Aberrometer 2 | | |
|---|---|---|---|---|---|---|
| X | M | J0 | J45 | M | J0 | J45 |
| [symbol] | −1.158685e+01 | −23.634670622 | −1.801281e+01 | −9.917428e+00 | −13.824630577 | −14.15287304 |
| [symbol] | −1.478691e+00 | −2.255383300 | −2.397918e+00 | −1.490140e+00 | −2.257999967 | −2.41899596 |
| [symbol] | −3.645794e+00 | −4.058939935 | −4.179863e+00 | −3.451811e+00 | −4.031299112 | −4.12621854 |
| [symbol] | 3.849776e−02 | −0.005832360 | 1.195480e−03 | −9.787282e−02 | −0.010296598 | 0.00105432 |
| [symbol] | 9.530755e−01 | 0.049540280 | 7.342407e−04 | 9.492652e−01 | 0.057330004 | −0.01627960 |
| [symbol] | 2.150168e−01 | 0.884831027 | −3.017279e−02 | 2.295930e−01 | 0.878258986 | −0.03860188 |
| [symbol] | −3.884270e−03 | −0.004837432 | 8.160447e−01 | −1.549439e−01 | 0.008366583 | 0.80318242 |
| [symbol] | * | −0.047771141 | −4.900606e−04 | * | −0.056451915 | 0.01814338 |
| [symbol] | −2.406537e−01 | * | 1.039228e−02 | −2.730474e−01 | * | 0.02477013 |
| [symbol] | −8.299017e−03 | 0.027505589 | * | 1.467214e−01 | 0.003420205 | * |
| [symbol] | −8.175954e−03 | 0.031644005 | 8.539891e−03 | −9.243794e−03 | 0.032050577 | 0.03769131 |
| [symbol] | 3.245316e−05 | 0.009592653 | 4.276131e−02 | −2.466715e−05 | 0.016408827 | 0.03787041 |
| [symbol] | 7.550834e−05 | −0.005829236 | −5.949418e−03 | 1.072269e−04 | −0.003381591 | −0.01119501 |

[symbol]

indicates data missing or illegible when filed

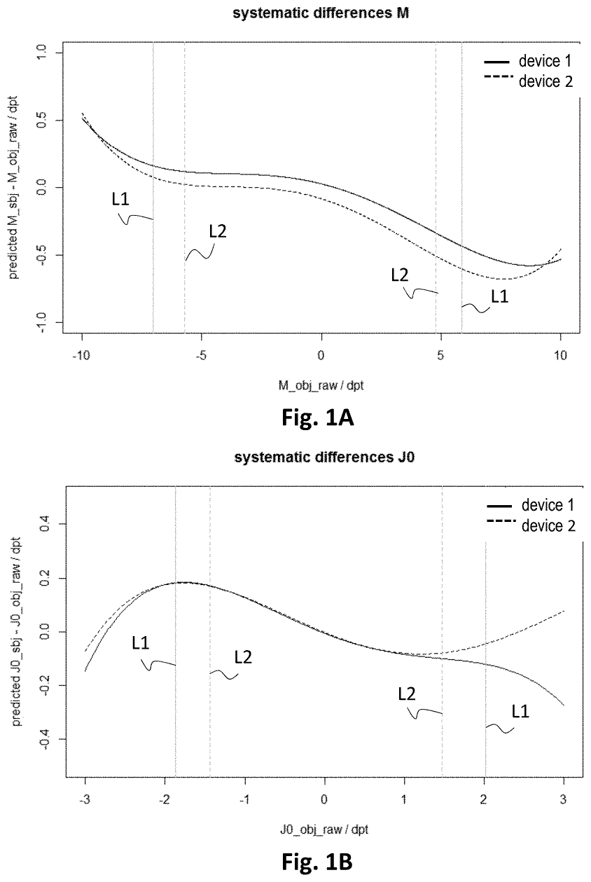

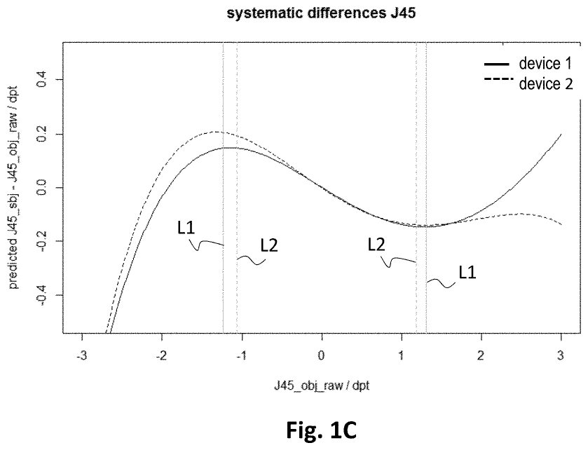

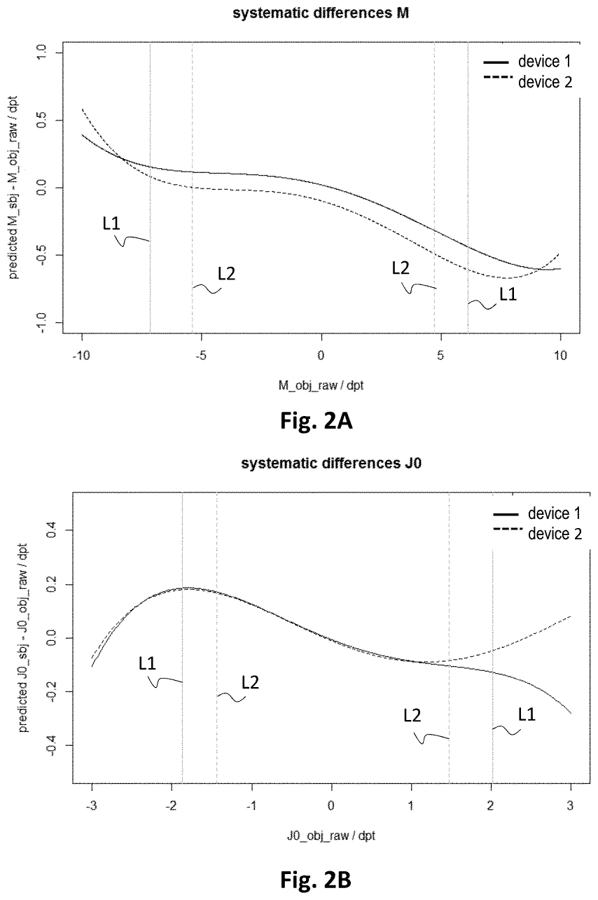

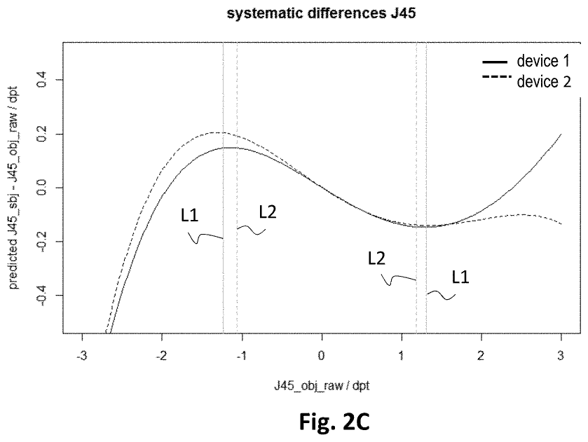

With the models thus parameterized, the systematic deviations of subjective and objective refraction in different apparatus models (such as two different aberrometer models) can be corrected. The course of the respective power vector components X when the objective power vector components Y≠X are set to 0 is shown in FIGS. 1 and 2.

FIGS. 1 and 2 show the systematic deviations of objective and subjective wavefronts for two different aberrometers (aberrometer 1: solid line, aberrometer 2: dashed line). Each power vector component was quantified with two different models. Model 1 (FIGS. 1 A to 1 C) does not include any influence of the subjective refraction, in model 2 (FIGS. 2A to 2B) the influence of the subjective refraction is included.

FIGS. 1A and 2A show the difference between the predicted value (predicted M_sbj) of the spherical equivalent M determined using subjective refraction and the value (M_obj_raw) of the spherical equivalent measured using objective refraction as a function of the value (M_obj_raw) of the spherical equivalent M measured using objective refraction.

FIGS. 1B and 2B show the difference between the predicted value (predicted J0_sbj) of the component J0 of the power vector of the subjective refraction and the measured value (J0_obj_raw) of the component J0 determined using objective refraction as a function of the measured value (J0_obj_raw) of the component J0 determined using objective refraction.

FIGS. 1C and 2C show the difference between the predicted value (predicted J0_sbj) of the component J45 of the power vector of subjective refraction and the measured value (J45_obj_raw) of the component J0 determined using objective refraction as a function of the measured value (J45_obj_raw) of component J0 determined using objective refraction.

The vertical lines L1 (model 1) and L2 (model 2) indicate the ranges in which there is a sufficiently high density of data in the data set, here about 50 eyes (left+right) per diopter of the respective power vector component.

As can be seen from FIGS. 1 and 2, in both aberrometer models, both in the first and in the second model, the spherical equivalent for hyperopes is subjectively lower than objectively. In the case of myopes, it is the other way around, and not as pronounced. Overall, a correction that is weaker in terms of magnitude can be seen in the subjective refraction. This is also the case with the astigmatic power vector components.

Correction of the Systematic Deviations of the Objective Refraction

If one assumes that the objective refraction is systematically wrong, the objective refraction can on average be matched to the subjective refraction using model 1 or model 2, for example. This is done by adding the power vector difference between subjective and objective refraction, ΔPᵢ, determined when fitting the model to a large number of data, to the power vector of the objective refraction \tilde{P}_{obj}. If the data was fitted to a model described in equation system 1, then

ΔP[\tilde{P}_{obj}]=P_{pred}[\tilde{P}_{obj}]−\tilde{P}_{obj}. (6)

When model 1 is used, the difference between subjective and objective refraction depends solely on the objective refraction:

P_{obj}=\tilde{P}_{obj}+ΔP[\tilde{P}_{obj}] (7)

In order to avoid overshoots of the model and thus a false correction, it is preferred to limit the corrections ΔP to a range in which sufficient data is available:

P_{obj}=\tilde{P}_{obj}+ΔP[B(\tilde{P}_{obj})] (7)

Here, the function B(.) limits the power vector \tilde{P}_{obj} to the range in which there is enough data. For example, B(.) can be implemented as a perpendicular projection onto the side surfaces of a simple box. Outside of range B, the change ΔP can be set to a constant value.

Table 3 shows possible limitations of the scope of the model. The power vector component X is mapped to max(min(X, Max_X), Min_X) by the limiting function B(.).

The limits are based on a data density of 50 measurements per diopter of the respective power vector component. Within the range shown in table 3, step 1 corrects the systematic differences relatively well. Outside the range, the change ΔP is kept constant.

Alternatively, other criteria can be used, such as, based on the data density divided by the determinant of the Jacobi matrix of the correction ΔP[\tilde{P}_{obj}] relative to the objective power vector, i.e. data density/det[∂ΔP[\tilde{P}_{obj}]/∂\tilde{P}_{obj}].

TABLE 3

| Power vector | Example 1 | | | Example 2 | | |
|---|---|---|---|---|---|---|
| component X | M/dpt | J0/dpt | J45/dpt | M/dpt | J0/dpt | J45/dpt |
| Min_X | −7.04 | −1.87 | −1.23 | −5.72 | −1.44 | −1.06 |
| Max_X | 5.89 | 2.02 | 1.31 | 4.76 | 1.47 | 1.19 |

Other limiting functions are also conceivable, such as the projection of any point in the range with no or with only a small number of data onto the edge of an iso-probability density area of the data in the space of the uncorrected, objective power vector. It is advantageous to carry out the projection along the gradient of the probability density of the data. In this case, the projection lines result from the solution of a linear differential equation and run through the power vector to be projected, which is a classic initial value problem. If the density of the data should be described by a multidimensional (e.g. 3-dimensional) distribution, the solution of the differential equation can even take place analytically. For other probability density functions, a numerical solution may be necessary. Once a projection line has been found, its intersection with the iso-probability area belonging to the desired data density can be carried out numerically with the aid of a 1-dimensional search along the projection line.

Other types of limitations of the correction are also possible. For example, it would be conceivable not to keep the change ΔP constant outside the limited range, but rather to allow it to change linearly as a function of the distance from the uncorrected, objective power vector \tilde{P}_{obj}j toward the edge of the boundary. This effectively corresponds to a piece-wise defined model that is a higher-order polynomial within the boundary and is linear outside the boundary. The transition at the boundary is to be selected such that the model can be continuously derived with respect to \tilde{P}_{obj}.

In the simplest case, the subjective refraction is not corrected and—as shown above—only the objective refraction is adapted to the subjective refraction in order to compensate for the quantified systematic differences between the two refraction methods.

Correction of Systematic Deviations in Subjective Refraction

However, it is also possible to adapt the subjective refraction to the objective refraction. In this case, to calculate the power vector of a corrected subjective refraction, P_{sub}, the systematic difference ΔP determined with the aid of the model is subtracted from the power vector of the original subjective refraction \tilde{P}_{sub}:

P_{sub}=\tilde{P}_{sub}+ΔP[\tilde{P}_{sub}]. (9)

This may be necessary, for example, if the systematic differences arise due to questionable refraction techniques, e.g. by omitting or changing the power of the cylinder refraction lens without adjusting the sphere accordingly by half the change in the power of the cylinder refraction lens, in order to keep the spherical equivalent constant.

Correction of the Systematic Deviations of the Objective and the Subjective Refraction

In general, it can also be advantageous to split the power vector of the systematic differences between subjective and objective power vectors, ΔP, into two parts, one of which, ΔP_{obj}, is used to correct the power vector of objective refraction, and the other, ΔP_{sub}, is used to correct the subjective refraction:

ΔP=ΔP_{sub}[\tilde{P}_{sub},\tilde{P}_{obj}]+ΔP_{obj}[\tilde{P}_{sub},\tilde{P}_{obj}]

P_{obj}=\tilde{P}_{obj}+ΔP_{obj}[\tilde{P}_{sub},\tilde{P}_{obj}]

P_{sub}=\tilde{P}_{sub}+ΔP[\tilde{P}_{sub}]. (10)

The differences ΔP_{obj} and ΔP_{sub} can depend both on the uncorrected, objective refraction, \tilde{P}_{obj}, and on the uncorrected, subjective refraction, \tilde{P}_{sub}. Here, P_{obj} is the corrected objective refraction and P_{sub} is the corrected subjective refraction.

The parts of the systematic differences can advantageously be divided such that terms of the model containing differences of uncorrected subjective and objective power vector components (e.g. terms proportional to [symbol] _{sub}− [symbol] _{obj} or a power thereof, which occur in the model of the spherical equivalent) can be used to correct the subjective refraction, as it is very likely the effect of a questionable refraction method such as the one mentioned above. The remaining terms (e.g. those that depend solely on the components of the power vector of the uncorrected objective refraction) can be used to correct the objective refraction.

It is also possible to split the corrections to the objective and subjective differences ΔP_{obj} and ΔP_{sub} with a real factor α common to all power vector components, or with real factors α, β, γ different for each power vector component:

P_{obj}=\tilde{P}_{obj}+αΔP_{obj}[\tilde{P}_{sub},\tilde{P}_{obj}]

P_{sub}=\tilde{P}_{sub}−(1−α)ΔP_{sub}[\tilde{P}_{sub},\tilde{P}_{obj}], (10)

or

$$\begin{matrix}{P}_{obj} = {\tilde{P}}_{obj} + [ \begin{matrix}α & 0 & 0 \\ 0 & β & 0 \\ 0 & 0 & γ\end{matrix} ] Δ {P}_{obj} [ {\tilde{P}}_{sub} , {\tilde{P}}_{obj} ] & ( 11 ) \\ {P}_{sub} = {\tilde{P}}_{sub} - ( 1 - [ \begin{matrix}α & 0 & 0 \\ 0 & β & 0 \\ 0 & 0 & γ\end{matrix} ] ) Δ {P}_{sub} [ {\tilde{P}}_{sub} , {\tilde{P}}_{obj} ] & \end{matrix}$$

However, this would only make sense if it were known that a third refraction method has no systematic errors or simply only has fewer systematic errors than the subjective refraction and the objective refraction, the systematic deviation of which has already been quantified. In addition, the systematic deviation of the subjective refraction and the objective refraction from the third refraction method would have to be proportional to the systematic deviations between the subjective and objective refraction.

The calculation of the corrected objective and subjective refractions can of course also be combined with a limitation of the correction.

If the corrected objective values are displayed to the refractionating person, for example on the aberrometer or auto refractometer, it is possible for the subjective refraction to be influenced by the objective measurement result. It can therefore also be advantageous to carry out the above-described method for determining the correction of the systematic differences between subjective and objective refraction several times, e.g. with data sets from half a year of orders. In this way, the influence of the subjective refraction by the representation of the objective refraction is gradually reduced.

Alternatively, it is also possible to select a model for determining the systematic differences that quantifies the proportion and extent of the influence. Such models can, for example, be set up on the basis of estimated values, or by evaluating studies with a relatively small number of refractionated and refractionating persons, part of whom has to create an objective refraction before the subjective refraction and the other part must not carry out an objective refraction. It is also possible for one and the same refractionating person to carry out subjective refractions of different persons both with and without a preceding objective refraction. In this case, however, it is also possible and, if necessary, supplementary to compare two distributions of a larger amount of refraction data (e.g. as power vectors) created by refractors who had no possibility to measure an objective refraction (first distribution), or by those who compellingly created an objective refraction (second distribution). Such data sets arise in large amounts during the ordering process for spectacle lenses and can be obtained and examined relatively easily without the need for special studies. If such a model is used, it is not absolutely necessary to repeat the method for determining the correction.

Instead of matching the systematic errors of the subjective and objective refractions as power vectors with each other, other representations of refraction errors can of course also be used, such as sphere, cylinder, axes or the Zernike decomposition of wave fronts.

In the case of a representation as a wavefront, the subjective refraction, preferably with the pupillary diameter present during the refraction, is converted into a wavefront (subjective wavefront). Methods for converting into a wavefront are known from the prior art. The higher-order aberrations generated by the refraction lenses could also be taken into account, even if these are generally low. Subsequently, a model for predicting the subjective wavefront from the objective wavefront can be adapted to the available data, i.e. to the subjective and objective wavefronts. It can be advantageous to standardize the representations, such as Zernike coefficients, before the analysis. The correction used for matching results analogously to the above-described method with power vectors from the difference of the predicted subjective wavefront and the objective wavefront.

Finally, a correction of the systematic deviations between objective and subjective refraction for objects at infinity (called distance refraction), as shown above, can also be applied to the near prescription, which is also called near refraction.

In the best case, the objective near refraction is present as a wavefront at the same distance d from the eye at which the corrections for the systematic deviations of the subjective and objective distance refraction are also present. If this is not the case, it must be converted into this distance according to the prior art. The same applies to the subjective near refraction.

If the near refraction is not present as a wavefront, but as the power of a spectacle lens, it should be noted that the near refraction itself must not be propagated for the conversion. Rather, a spherical wavefront emanating from a point at the object distance belonging to the near refraction, which has been refracted by an imaginary refraction lens including the near refraction, must be propagated at the distance belonging to the near refraction (so-called corneal vertex distance) from the eye.

The correction of the systematic deviations can now be applied to the thus wavefront calculated. If one wishes to obtain a corrected near refraction again—but this time at the distance d to the eye—the difference to a spherical reference wavefront must be calculated, which was propagated starting from a point in the refraction distance to the same distance d.

Step 2—Combination of the Corrected Wavefronts or the Corrected Components of the Refraction

The power vectors of the subjective and objective wavefronts P_{sub} and P_{obj} are averaged in a weighted manner after the correction of systematic differences. The weights of the spherical equivalent M are of great importance, since the risk of refraction that is too myopic changes depending on the accommodation ability and especially when it is restricted as a result of the aging process of the eye lens. The weights of the astigmatic components, i.e. for J0 and J45, can e.g. be set to 0.7 for the subjective refraction, and the corresponding objective components to 0.3.

According to one example, advantageous weights of the spherical equivalent are proposed, which enable a particularly accurate estimate of the vision disorder of the spectacle wearer. The motivation for the proposed weights of the spherical equivalent arose from the following thought:

In cases where objective and subjective spherical equivalents are consistent, both data sources should be used. If the measurements are not consistent, the weights should be adjusted depending on the risk of a measurement that is too myopic: The lower the addition, the higher the risk of a measurement that is too myopic (both for subjective and objective refraction). In this case, the more positive spherical equivalent is given a higher weight. The higher the addition, the lower the risk of a measurement that is too myopic, so that a large deviation in the subjective and objective spherical equivalents is likely to have other reasons. For this reason, the subjective measurement is preferably given a high weighting in these cases, since the refracted person has already tested a corresponding lens during the refraction.

Calculating the Weights

The weights can be calculated or determined based on the difference between the objectively and subjectively determined spherical equivalents

ΔM=M_{sub}−M_{obj}, (12)

and depending on the accommodation ability that can be determined from the addition,

Akk=(Add|A₁^{N}), (13)

### Here:

M_{sub}: spherical equivalent from subjective refraction
M_{obj}: spherical equivalent from objective refraction
AAk: accommodation ability calculated from the addition
Add: addition measured during refraction, or the prescribed addition
A₁^{N}: reciprocal object distance in the determination of the addition (negative sign convention, i.e.

A₁^{N}<0,z·B. for an object at a distance of 40 cm, A₁^{N}=−2.5 Dpt)

To calculate the weights g_{sub}^{M}(ΔM,Akk) of the subjectively determined spherical equivalent M_{sub}, auxiliary weights g_{sub}^{M}(ΔM,Akk₁) and g_{sub}^{M}(ΔM,Akk₂) can be determined as a function of ΔM:

$${g}_{sub}^{M} ( Δ M , {Akk}_{i} ) = \{ \begin{matrix}{g}_{sub}^{M} ( Δ {M}_{- 2} , {Akk}_{i} ) & f ü r Δ M \leq Δ {M}_{- 2} \\ {g}_{sub}^{M} ( 0 , {Akk}_{i} ) \cdot \frac{Δ M - Δ {M}_{- 2}}{Δ {M}_{- 1} - Δ {M}_{- 2}} + {g}_{sub}^{M} ( Δ {M}_{- 2} , {Akk}_{i} ) \cdot \frac{Δ {M}_{- 1} - Δ M}{Δ {M}_{- 1} - Δ {M}_{- 2}} & f ü r Δ {M}_{- 2} < Δ M < Δ {M}_{- 1} \\ {g}_{sub}^{M} ( 0 , {Akk}_{i} ) & f ü r Δ {M}_{- 1} \leq Δ M \leq Δ {M}_{+ 1} \\ {g}_{sub}^{M} ( 0 , {Akk}_{i} ) \cdot \frac{Δ {M}_{+ 2} - Δ M}{Δ {M}_{+ 2} - Δ {M}_{+ 1}} + {g}_{sub}^{M} ( Δ {M}_{+ 2} , {Akk}_{i} ) \cdot \frac{Δ M - Δ {M}_{+ 1}}{Δ {M}_{+ 2} - Δ {M}_{+ 1}} & f ü r Δ {M}_{+ 1} < Δ M < Δ {M}_{+ 2} \\ {g}_{sub}^{M} ( Δ {M}_{+ 2} , {Akk}_{i} ) & f ü r Δ {M}_{+ 2} \leq Δ M ,\end{matrix} \qquad ( 14 )$$

where 1 or 2 is substituted for f.

The subjective weight is obtained by linearly interpolating the auxiliary weights in the range between Akk₁ and Akk₂:

$${g}_{sub}^{M} ( Δ M , Akk ) = \{ \begin{matrix}{g}_{sub}^{M} ( Δ M , {Akk}_{1} ) & f ü r Akk \leq {Akk}_{1} \\ {g}_{sub}^{M} ( Δ M , {Akk}_{1} ) \cdot \frac{Δ {kk}_{2} - Akk}{{Akk}_{2} - {Akk}_{1}} + {g}_{sub}^{M} ( Δ M , {Akk}_{2} ) \cdot \frac{Akk - {Akk}_{1}}{{Akk}_{2} - {Akk}_{1}} & f ü r {Akk}_{1} < Akk < {Akk}_{2} \\ {g}_{sub}^{M} ( Δ M , {Akk}_{2} ) & f ü r {Akk}_{2} \leq Akk ,\end{matrix} \qquad ( 15 )$$

The weights of the objectively determined spherical equivalent can be calculated from the weights of the subjectively determined spherical equivalent by g_{obj}^{M}(ΔM,Akk)=1−g_{sub}^{M}(ΔM,Akk).

Ranges for the support points and their weights are listed below:

−1.5 Dpt≤ΔM₋₂≤0.5 Dpt

−1.0 Dpt≤ΔM₋₁≤0.25 Dpt

0.25 Dpt≤ΔM₊₁≤1.0 Dpt

0.5 Dpt≤ΔM₊₂≤1.5 Dpt

where ΔM₋₂<ΔM₋₁<ΔM₊₁<ΔM₊₂.

0 Dpt≤Akk₁≤1.25 Dpt.

1.0 Dpt≤Akk₂≤2.75 Dpt

where Akk₁<Akk₂.

The weights at the support points can be selected from the following ranges:

0.8≤g_{sub}^{M}(ΔM₋₂,Akk₁)≤1

0.3≤g_{sub}^{M}(0,Akk₁)≤0.7

0.8≤g_{sub}^{M}(ΔM₊₂,Akk₁)≤1

0≤g_{sub}^{M}(ΔM₋₂,Akk₂)≤0.5

0.3≤g_{sub}^{M}(0,Akk₂)≤0.7

0.8≤g_{sub}^{M}(ΔM₊₂,Akk₂)≤1

Examples of the choice of weights and support points:

### Example 1

−ΔM₋₂=ΔM₊₂=1.0 Dpt

−ΔM₋₁=ΔM₊₁=0.5 Dpt

Akk₁=0 Dpt

Akk₂=1.75 Dpt

g_{sub}^{M}(ΔM₋₂,Akk₁)=0.95

g_{sub}^{M}(0,Akk₁)=0.75

g_{sub}^{M}(ΔM₊₂,Akk₁)=0.95

g_{sub}^{M}(ΔM₋₂,Akk₂)=0.5

g_{sub}^{M}(0,Akk₂)=0.75

g_{sub}^{M}(ΔM₊₂,Akk₂)=0.95

### Example 2

−ΔM₋₂=ΔM₊₂=0 Dpt

−ΔM₋₁=ΔM₊₁=0 Dpt

Akk₁=0 Dpt

Akk₂=1.75 Dpt

g_{sub}^{M}(ΔM₋₂,Akk₁)=0.5

g_{sub}^{M}(0,Akk₁)=0.75

g_{sub}^{M}(ΔM₊₂,Akk₁)=1

g_{sub}^{M}(ΔM₋₂,Akk₂)=0.75

g_{sub}^{M}(0,Akk₂)=0.75

g_{sub}^{M}(ΔM₊₂,Akk₂)=0.75

### Example 3

−ΔM₋₂=ΔM₊₂=1.5 Dpt

−ΔM₋₁=ΔM₊₁=0.75 Dpt

Akk₁=0.5 Dpt

Akk₂=2.0 Dpt

g_{sub}^{M}(ΔM₋₂,Akk₁)=1

g_{sub}^{M}(0,Akk₁)=0.5

g_{sub}^{M}(ΔM₊₂,Akk₁)=1

g_{sub}^{M}(ΔM₋₂,Akk₂)=0

g_{sub}^{M}(0,Akk₂)=0.5

g_{sub}^{M}(ΔM₊₂,Akk₂)−1

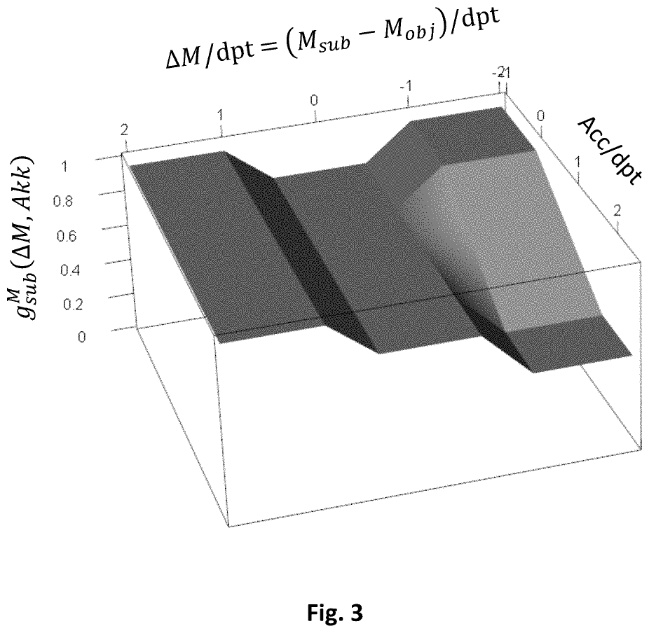

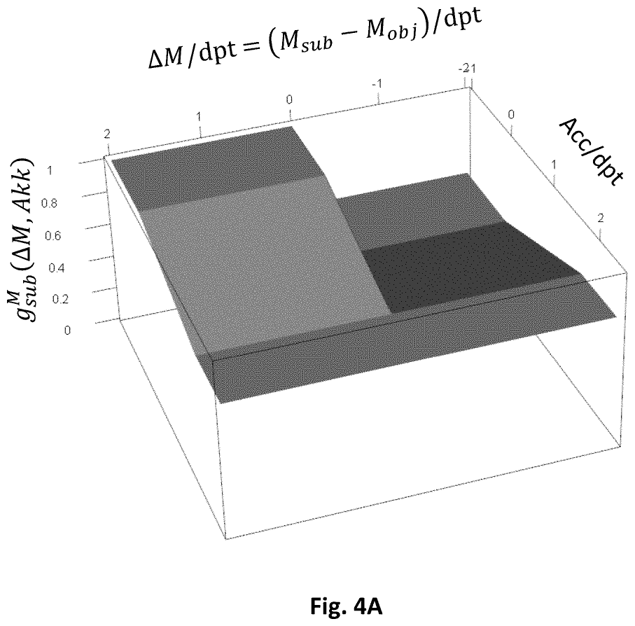

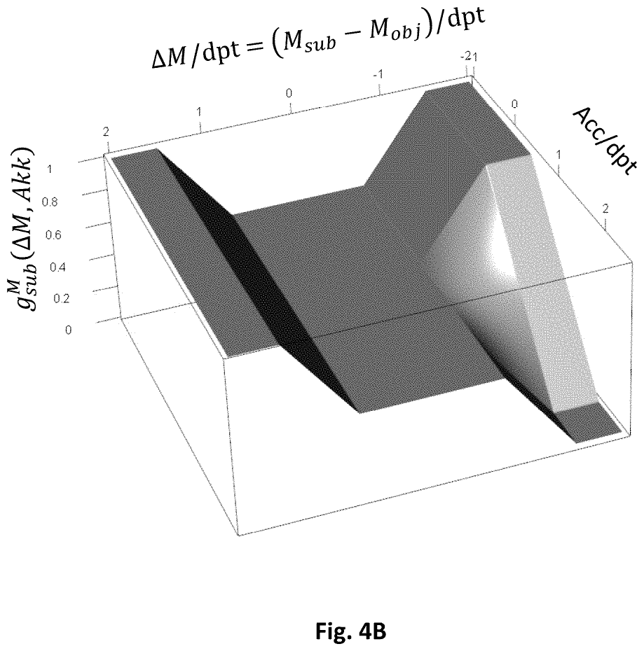

FIG. 3 shows the weights of the subjective spherical equivalent g_{sub}^{M} according to example 1. FIG. 4A shows the weights of the subjective spherical equivalent g_{sub}^{M} according to example 2. FIG. 4C and shows the weights of the subjective spherical equivalent g_{sub}^{M} according to example 3. The weights shown in FIG. 3 are more advantageous than the weights shown in FIG. 4A with respect to reducing the statistical measurement inaccuracies of the subjective and/or objective measurement.

The weights according to example 1 (FIG. 3), example 2 (FIG. 4A) and example 3 (FIG. 4B) depend on the addition and the difference (or the dispartiy) of the spherical equivalents ΔM=M_{sub}−M_{obj} corrected in step 1. The functions consist of several plateaus of constant weight, between which, as described above, linear interpolation is carried out.

An essential difference in the choice of weights according to example 1 compared to the choice of weights according to example 2 is that in the range in which ΔM is approximately normally distributed, here e.g. in the range 0.5 Dpt<ΔM<+0.5 Dpt, a plateau with a weight of e.g. g_{sub}^{M}=0.75 is introduced. Most measurements fall into this range. Because of the approximately normally distributed difference g_{sub}^{M}=0.75, it can be assumed that the spherical equivalents of the subjective and objective refraction are not contradictory, so that the spherical equivalent of the objective refraction can have a relatively high weight, e.g. 0.25. Outside this range, with a high addition, the subjective weight rises to a very high value, for example to 0.95 or even 1.0, regardless of the sign of the difference ΔM, since here accommodation is very unlikely. In the case of low additions, the subjective refraction is only weighted very high if it was more hyperoperic than the objective one. If it is myopic, the subjective weight will be reduced to a low value, for example to 0.5.

The change in the weights depending on the sign of ΔM is the second fundamental difference between the two types of weighting: in the method according to the first example this takes place with low additions, in the method according to the second example this is the case with high additions.

In order to combine near prescriptions, the corrected wavefronts belonging to the subjective and objective refraction can be combined analogously to the distance refraction. However, due to the greater measurement uncertainty, it is advantageous to weight the objective refraction only weakly, as described in the prior art.

In the following, on the basis of a (relatively small) data set (reference data set), the changes that result compared to the previously known method for the subjective, objective and combined refraction are evaluated. In the following example the subjective refraction is not changed and is therefore not shown there.

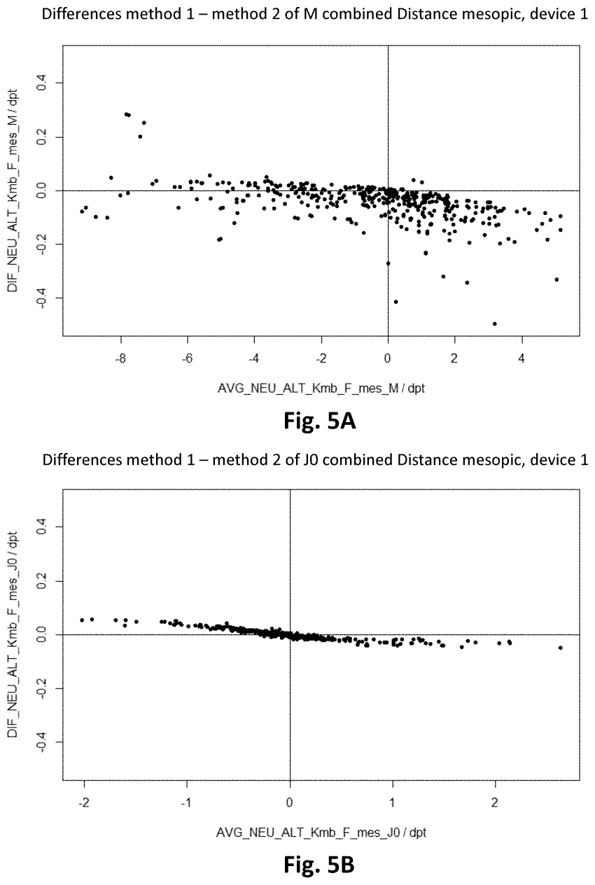

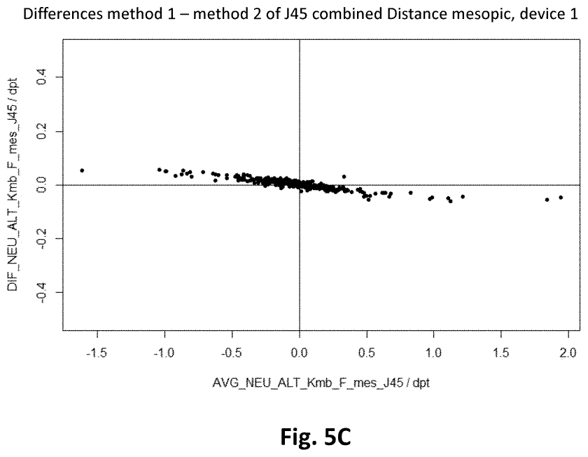

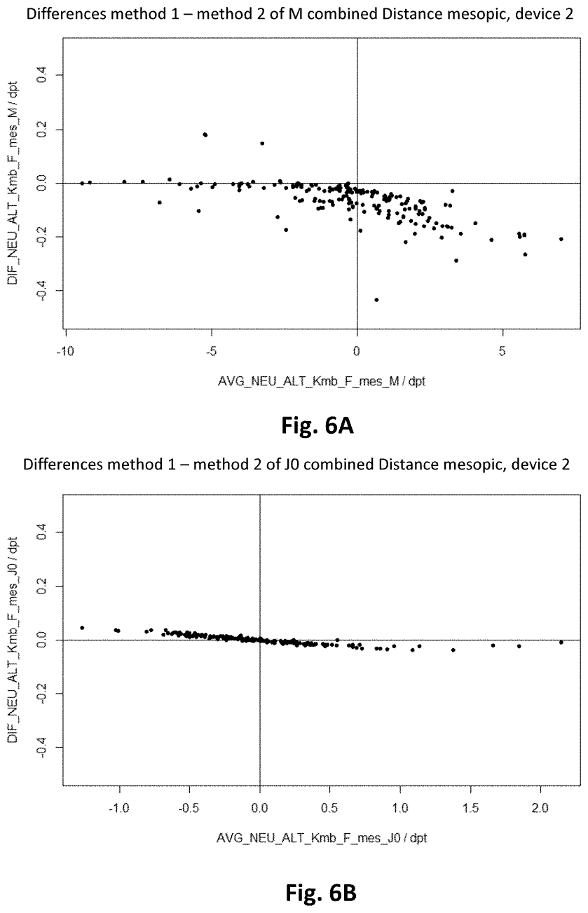

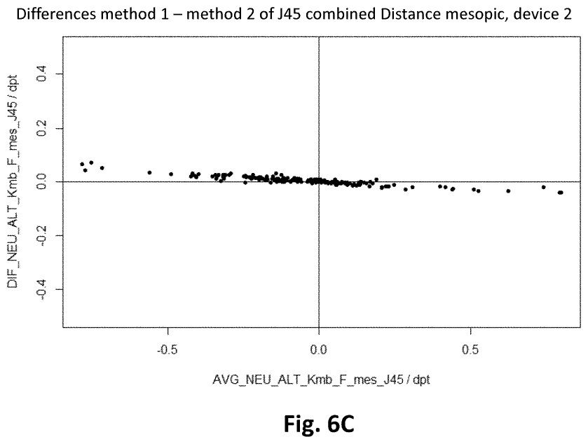

FIGS. 5 and 6 show the change in the estimated value of the vision disorder komb_F) calculated according to two different methods (with a pupil interpolated between a photopic and a mesopic pupil for two different devices. In particular, FIGS. 5 and 6 show the difference in the values obtained with a first method comprising steps 1 and 2 described above and with the weights according to example 1 and a second method carried out without matching the objective to the subjective refraction on average (i.e. without step 1, only step 2) and with the weights according to example 22. The objective measurement values are obtained with two different apparatuses (aberrometers), aberrometers 1 and 2, with FIG. 5 showing the results for the first aberrometer and FIG. 6 showing the results for the second aberrometer. FIGS. 5A and 6A show the differences of the spherical equivalent M, FIGS. 5B and 6B show the differences of the component J0, and FIGS. 5C and 6C show the differences for component J45.

The objective refraction for a pupil estimated from two other pupils (a photopic pupil and a mesopic pupil) shows the expected differences resulting from step 1 of the method. The refraction of the interpolated pupil, combined from the subjective corrected refraction and the objective corrected refraction, also shows the expected changes, which mainly result from matching objective to subjective.

The “outliers” in the illustrations are all non-presbyopes, for which there is the risk of a refraction that is too myopic. With them, the more positive refraction is weighted highly.

A procedure to generally weight the refraction with more plus more highly in the case of a higher addition would, if systematic differences between the two refractions have already been corrected, lead to a systematic shift of the combined refraction toward Plus even without “outliers”, such as device myopia. This is undesirable because a refraction that is too hyperopic cannot be compensated for by accommodation or by lowering the gaze in the progressive lens. Since device myopia is unlikely with high additions and can only occur with low additions, the new method with step 1) and possibly step 2) with the weights shown in FIG. 3 leads to a better combined refraction in this aspect.

The elimination of the systematic errors of the objective refraction (step 1) together with an addition-dependent weighting of the refraction (step 2) is, in comparison with an alternative method in which the systematically deviating refraction is only weighted low but not shifted, more advantageous in particular because the mean subjective refraction can be calculated with high accuracy from the objective refraction even if systematic differences occur between the two types of refraction. Instead, the choice of weights should ideally take place based on the trustworthiness of the refraction methods already corrected.

Overall, the proposed method with steps 1 and 2 leads to a decrease in the likelihood of reclamation for spectacle lenses in which both subjective and objective refraction are included in the calculation, especially in the power ranges in which aberrometers systematically measure differently than the subjective refraction.

The following are examples of series of spectacle lenses that can be calculated and produced using the method described above.

Spectacle Lens Series B1:

A series of spectacle lenses for correcting the vision disorder of a plurality of eyes, which comprises at least a first spectacle lens A having a first power P_A in at least one reference point, which corrects a vision disorder of a first eye characterized by at least
a first measurement value P_A1 obtained using a measuring device of a first type and consisting of several components, and
at least a second measurement value P_A2 obtained using a measuring device of a second type and consisting of several components, wherein
the first measurement value P_A1 of the first lens determined with a measuring device of the first type and the second measurement value P_A2 of the first lens determined with a measuring device of the second type differ in at least one component X;
the component X of the first power P_A present in the reference point of the first spectacle lens is closer to the component X of the measurement value P_A1 or P_A2 of the first spectacle lens that whose measuring device has the lower inaccuracy in the measurement of the component X, and wherein
the components X are a component of a wavefront representation of the vision disorder, its linear combination or variables derived therefrom.

Spectacle Lens Series B2:

A series of spectacle lenses according to series B1, wherein further the component X of the first power P_A present in the reference point of the first spectacle lens and the component X of the first measurement value of the first eye P_A1 are almost identical, although the first measurement value of the first eye P_A1 and the second measurement value of the first eye P_A2 differ in at least the component X.

Spectacle Lens Series B3:

A series of spectacle lenses according to series B1 or B2, which
comprises at least a second spectacle lens B, which has a second power P_B at least in a reference point identified identically in comparison with the first spectacle lens, which corrects a vision disorder of the second eye characterized by at least a first measurement value P_B1 determined using a measuring device of the first type and consisting of several components and at least a second measurement value P_B2 determined using a measuring device of the second type and consisting of several components, and
comprises at least a third spectacle lens C, which has a second power P_C at least in a reference point identified identically in comparison with the first spectacle lens, which corrects a vision disorder of the third eye characterized by at least a first measurement value P_C1 determined using a measuring device of the first type and consisting of several components and at least a second measurement value P_C2 determined using a measuring device of the second type and consisting of several components, wherein
the first measurement values P_A1, P_B1 and P_C1 of the first, second and thirds eyes determined with the measuring device of the first type are identical in terms of components,
the components X of the second measurement values P_A2, P_B2 and P_C2 of the first, second and thirds eyes determined with the measuring device of the second type all differ pairwise,
the component X of the first power P_A present in the reference point of the first spectacle lens and
the component X of the first measurement value of the first eye P_A1 are almost identical, and wherein
for the components X of the power of the i^{th} spectacle lens present in the reference point, X_i, and for the components X of the second measurement values of the i^{th} eyes, X_i2, the following relationships apply:

(X_B−X_A)/(X_B2−X_A2) unequal (X_C−X_A)/(X_C2−X_A2)

abs(X_B2−X_A2)<abs(X_C2−X_A2)

signum(X_B2−X_A2)=signum(X_C2−X_A2).

Spectacle Lens Series B4:

A series of spectacle lenses according to series B3, wherein:
the first, second and third spectacle lenses are single vision lenses or progressive lenses with the same addition Add,
where Add<=1.5 dpt, and wherein
for the components X of the power of the i^{th} spectacle lens present in the reference point, X_i, and for the components X of the second measurement values of the i^{th} eyes, X_i2, the following relationships apply:

(X_B−X_A)/(X_B2−X_A2)<(X_C−X_A)/(X_C2−X_A2) if X_B2−X_A2>0,X_C2−X_A2>0, and

(X_B−X_A)/(X_B2−X_A2)>(X_C−X_A)/(X_C2−X_A2) if X_B2−X_A2<0,X_C2−X_A2<0.

Spectacle Lens Series B5:

A series of spectacle lenses according to series B3, wherein

the first, second and third spectacle lenses are progressive lenses with the same addition Add,
where Add<=2 dpt, and wherein
for the components X of the power of the i^{th} spectacle lens present at the reference point, X_i, and for the components X of the second measurement values of the i^{th} eyes, X_i2, the following relationships apply:

(X_B−X_A)/(X_B2−X_A2)>(X_C−X_A)/(X_C2−X_A2) if X_B2−X_A2>0,X_C2−X_A2>0, and

(X_B−X_A)/(X_B2−X_A2)>(X_C−X_A)/(X_C2−X_A2) if X_B2−X_A2<0,X_C2−X_A2<0.

Spectacle Lens Series B6:

A series of spectacle lenses according to one of series B 1 to B5, wherein the measuring device of the first type can be used for subjective refraction.

Spectacle Lens Series B7:

A series of spectacle lenses according to one of the series B1 to B6, wherein the measuring device of the second type can be used to determine the objective refraction.

Spectacle Lens Series B8:

A series of spectacle lenses according to one of the series B1 to B6, which:
comprises at least a fourth spectacle lens D, which has a fourth power P_D at least in a reference point identified identically in comparison with the first spectacle lens, which corrects a vision disorder of a fourth eye characterized by at least a first measurement value P_D1 obtained using a measuring device of the first type and consisting of several components and at least a second measurement value P_D2 obtained using a measuring device of the second type and consisting of several components, and
comprises at least a fifth spectacle lens E, which has a fifth power P_E at least in a reference point identified identically in comparison with the first spectacle lens, which corrects a vision disorder of a fourth eye characterized by at least a first measurement value P_E1 determined using a measuring device of the first type and consisting of several components and at least a second measurement value P_E2 determined using a measuring device of the second type and consisting of several components, and wherein
the first measurement values P_A1, P_D1 and P_E1 of the first, fourth and fifth eyes determined with the measuring device of the first type are identical in terms of components,
the components X of the second measurement values P_A2, P_D2 and P_E2 of the first, fourth and fifth eyes determined with the measuring device of the second type all differ pairwise,
the component X of the first power P_A present in the reference point of the first spectacle lens and the component X of the first measurement value of the first eye P_A1 are almost identical, and wherein
for the components X of the power of the i^{th} spectacle lens present in the reference point, X_i, and for the components X of the second measurement values of the i^{th} eyes, X_i2, the following relationships apply:

X_D2−X_A2>0,X_E2−X_A2<0,X_D−X_A>0 and X_E−X_A<0.

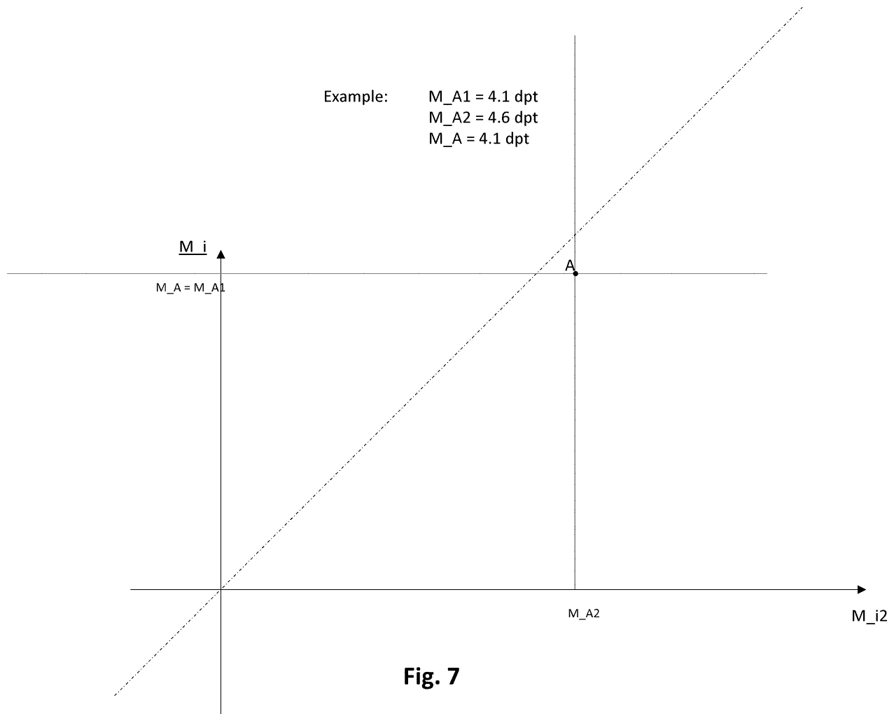

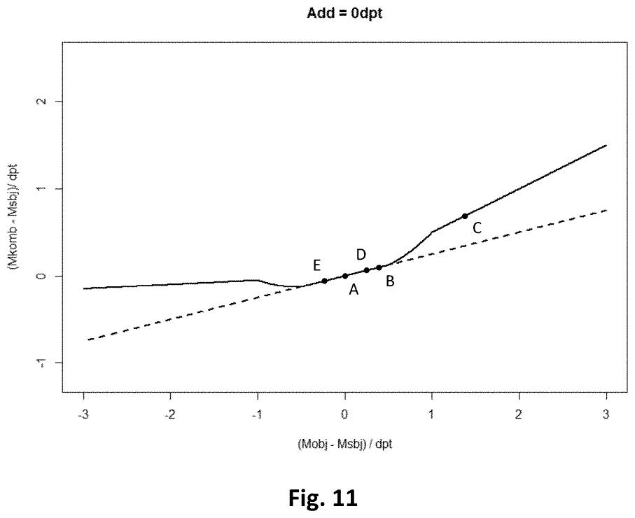

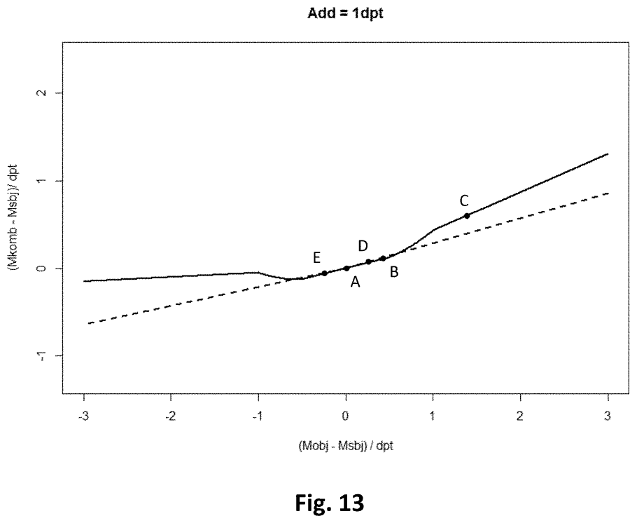

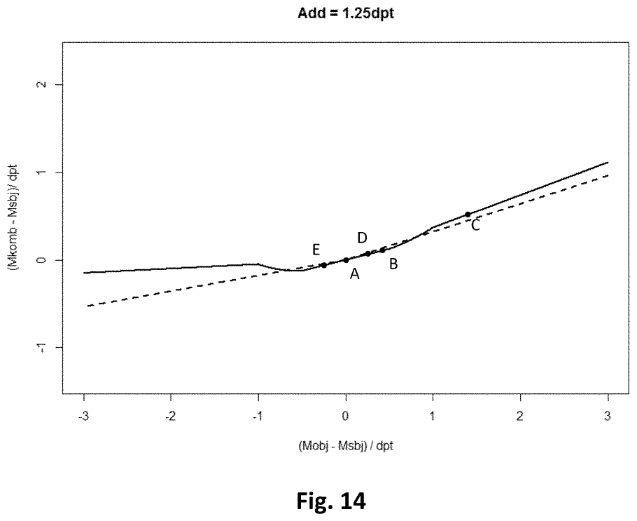

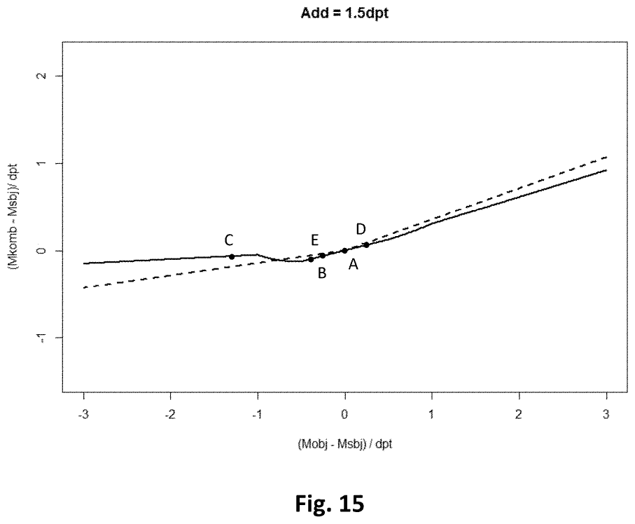

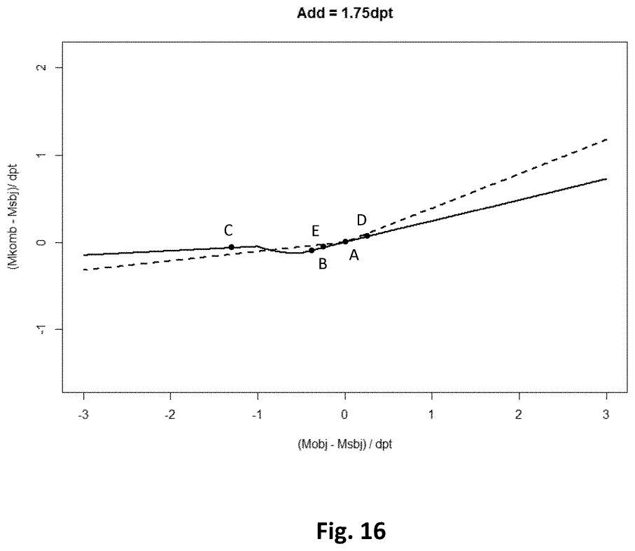

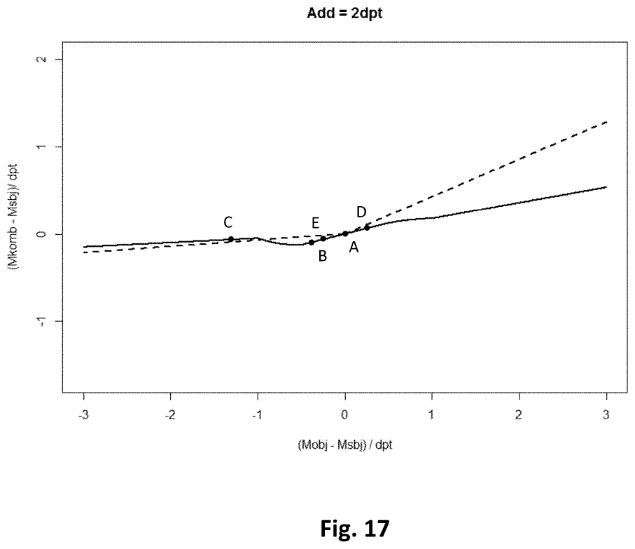

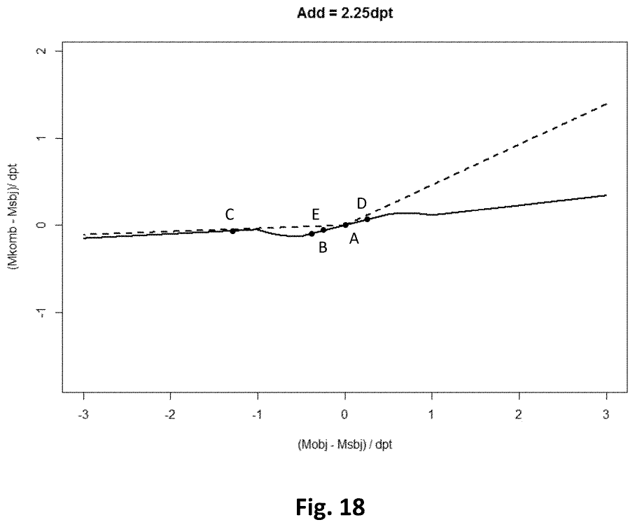

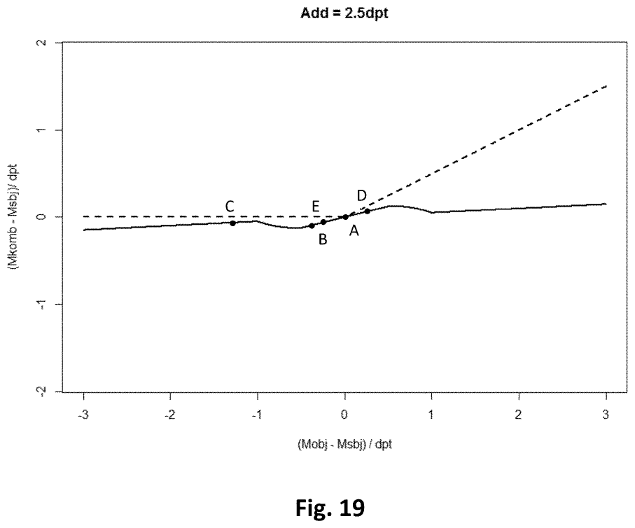

FIGS. 7 to 10 show individual representative spectacle lenses of the above series of spectacle lenses. The spectacle lenses shown have selected properties that allow properties of the ΔM-dependent weights of FIGS. 11 to 20 to be specified on the basis of 1, 3 or 5 lenses in a series, regardless of whether or not a correction of the systematic errors has been performed. The spectacle lenses lying on the solid or dashed lines in the detailed figures have the same subjective spherical equivalent (on the line shown, the subjective spherical equivalent is M_A1=M_A=4.1 dpt). The solid or dashed lines relate to support points and weights from examples 1 and 2, respectively.

FIG. 7 relates to a spectacle lens A of the spectacle lens series B2, which was calculated using a method comprising step 1. The spectacle lens A shown in FIG. 7 has the subjectively measured spherical equivalent in the reference point (i.e. M_A=MA_1=4.1 dpt), although the objectively measured spherical equivalent (M_A2=4.6 dpt) substantially differs from the subjective measured spherical equivalent (M_A 1=4, 1 dpt). The objectively measured spherical equivalent was nevertheless taken into account in the calculation.

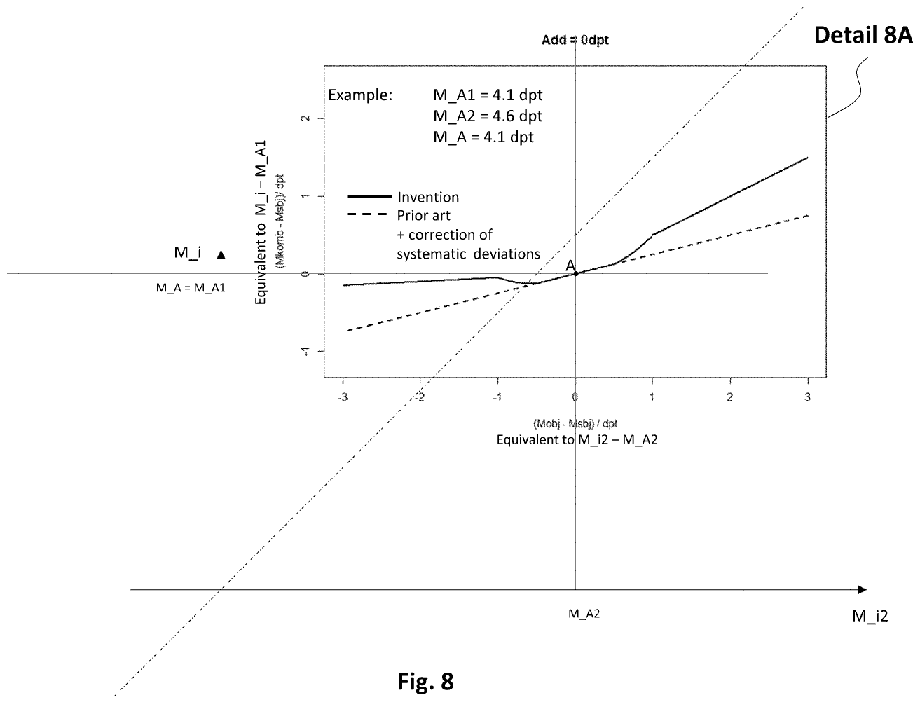

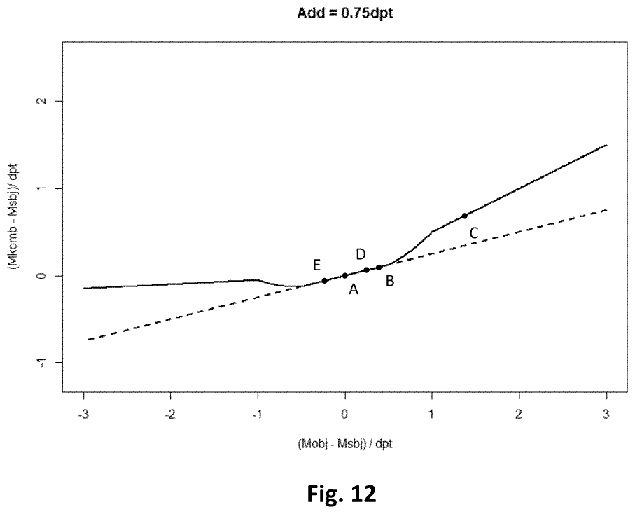

FIG. 8 relates to another spectacle lens A of a spectacle lens series B2, which was calculated using a method comprising steps 1 and 2. The spectacle lens shown in FIG. 8A has similar properties to the spectacle lens shown in FIG. 7. The detail 8A corresponds to FIGS. 11 and 12.

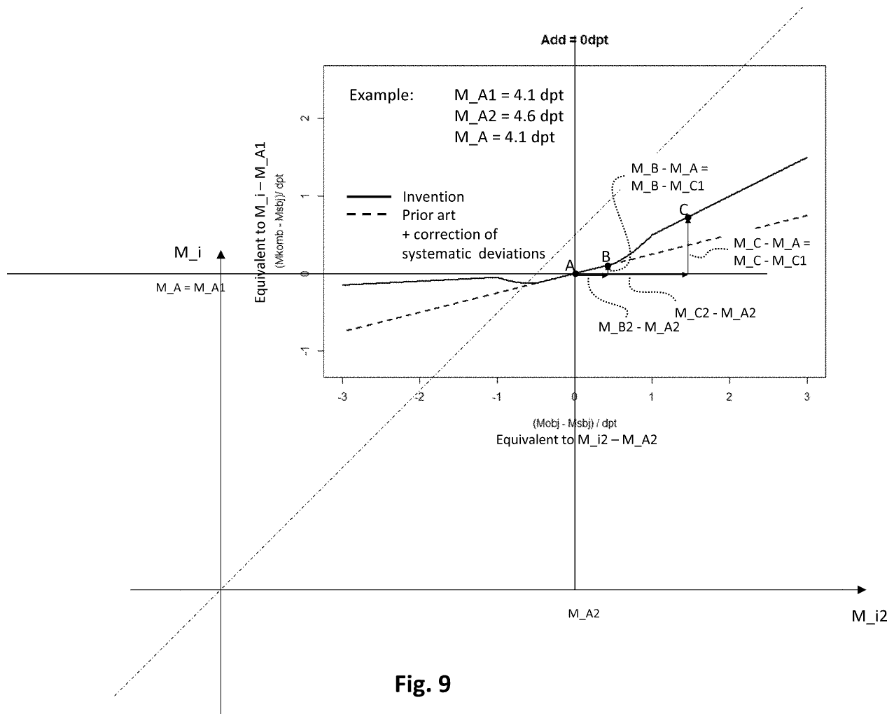

FIG. 9 relates to 3 spectacle lenses A, B, C of the spectacle lens series B3, which were calculated using a method comprising steps 1 and 2. Here, it is characteristic that all lenses have the same subjective spherical equivalent and that the iso-line of the same spherical equivalent has different slopes at least in one of the intervals Mobj<M_A2 and Mobj>M_A2—this is expressed by the relationships between the spherical equivalents of the lenses A, B and C measured and present in the reference point.

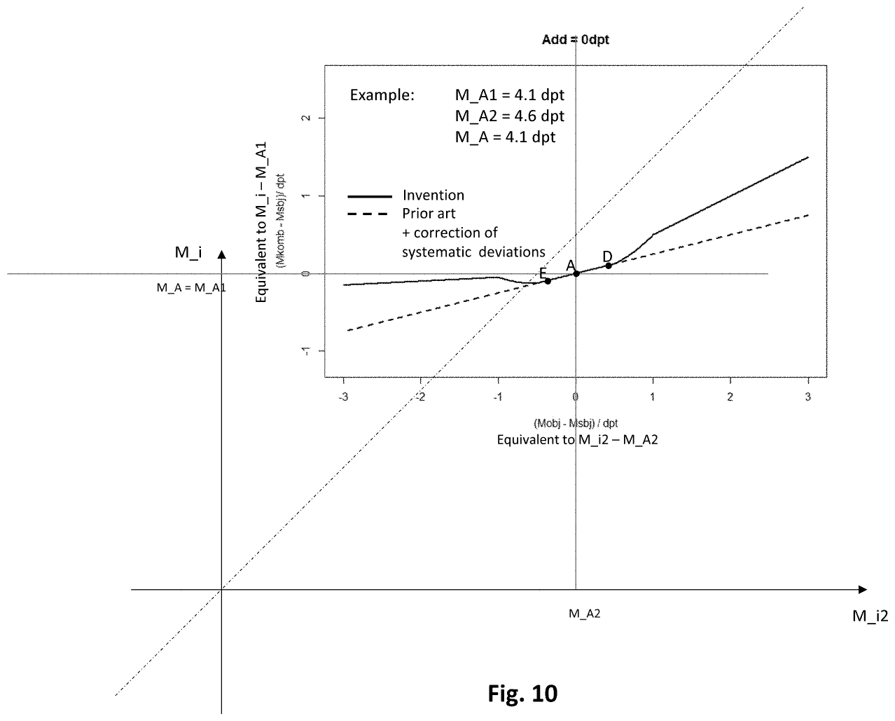

FIG. 10 relates to spectacle lenses A, D and E of the spectacle lens series B8. What is characteristic here is the slope of the iso-line of identical spherical equivalent, which is identical on both sides of M_A2, which is expressed by the relationships between the spherical equivalents of the lenses A, D and E measured and present in the reference point.

FIGS. 11 to 19 show the difference (Mkomb−Msbj) between an estimated value (Mkomb) of the spherical equivalent and a measured subjective spherical equivalent (Msbj) as a function of the difference between a measured objective spherical equivalent (Mobj) and a measured subjective spherical equivalent for different additions Add. “Mkomb” denotes the combined spherical equivalent, i.e. an estimated value of the spherical equivalent calculated according to an exemplary method comprising steps 1 and/or 2. On the x-axis there is the difference of the objective spherical equivalent Mobj (e.g. M_B2 or M_C2) for a spectacle lens (e.g. a spectacle lens B or C) minus the objective spherical equivalent Mobj=Msbj, in which the combined spherical equivalent is equal to the subjective spherical equivalent (e.g. M_B2−M_A2 or MC_2−M_A2). FIGS. 11 to 19 relate to additions that were determined at the standard object distance of 40 cm (corresponds to A₁^{N}=2.5 Dpt).

The solid or dashed lines show the cases in which the combined spherical equivalent Mkomb was obtained according to an exemplary method comprising step 2 with support points and weights of example 1 (solid) and example 2 (dashed). The combined spherical equivalent “Mkomb” thus represents the estimated value of the vision disorder according to an exemplary method comprising step 2 or steps 1 and 2.

1-24. (canceled)

25. A computer-implemented method for determining the vision disorder of an eye of a spectacle wearer, comprising:
providing measurement values from a first and a second measurement of the vision disorder of the eye of the spectacle wearer; and
calculating an estimated value for the vision disorder of the eye of the spectacle wearer based on the measurement values from the first and the second measurement, wherein measurement inaccuracies of the first and the second measurements of the vision disorder are taken into account in the calculation of the estimated value of the vision disorder.

26. The method according to claim 25, wherein the measurement inaccuracies comprise a statistical and/or a systematic deviation between the measurement values from the first measurement and the measurement values from the second measurement.

27. The method according to claim 25, wherein
the first measurement of the vision disorder of the eye is an objective refraction, and/or
the second measurement of the vision disorder of the eye is a subjective refraction.

28. The method according to claim 27, further comprising:
determining the measurement inaccuracies of the first and second measurements using statistical analysis of a data set with a plurality of reference measurement values, which include a first measurement and a second measurement of the vision disorder of the eyes of different spectacle wearers.

29. The method according to claim 28, wherein the determining the measurement inaccuracies of the first measurement and the second measurements comprises:
setting a model for the measurement values of the second measurement as a sum of a predicted measurement value and a random variable, wherein the predicted measurement value is modeled as a parametric function of the measurement value of the first measurement and optionally a part of the measurement value of the second measurement;
specifying the parameters of the parametric function by adapting the model to the reference measurements contained in the data set while maximizing the probability distribution of the random variables in the parameter space of the model; and
determining a systematic deviation of the first measurement from the second measurement on the basis of the predicted measurement.

30. The method according to claim 29, wherein the predicted measurement is a predicted refraction, which can be modeled by one of the following parametric functions:
$$\begin{matrix}{M}_{pred} ( {\tilde{M}}_{obj} , {obj}_{} , {obj}_{} ) = {\sum }_{i = 0}^{4} {a}_{M , i}^{M} {\tilde{M}}_{obj}^{i} + {a}_{J 0 , 1}^{M} {obj}_{} + {a}_{J 45 , 1}^{M} {obj}_{} & Model 1 \\ J {0}_{pred} ( {\tilde{M}}_{obj} , {obj}_{} , {obj}_{} ) = {a}_{M , 1}^{J 0} {\tilde{M}}_{obj} + {\sum }_{i = 0}^{4} {a}_{J 0 , i}^{J 0} {obj}_{i}^{} + {a}_{J 45 , 1}^{J 0} {obj}_{} & \\ J {45}_{pred} ( {\tilde{M}}_{obj} , {obj}_{} , {obj}_{} ) = {a}_{M , 1}^{J 45} {\tilde{M}}_{obj} + {a}_{J 0 , 1}^{J 45} {obj}_{} + {\sum }_{i = 0}^{4} {a}_{J 45 , i}^{J 45} {obj}_{i}^{} & \\ or & \\ {M}_{pred} ( {\tilde{M}}_{obj} , {obj}_{} , {obj}_{} , {sub}_{} , {sub}_{} ) = {\sum }_{i = 0}^{4} {a}_{M , i}^{M} {\tilde{M}}_{obj}^{i} + {a}_{J 0 , 1}^{M} {obj}_{} + {a}_{J 45 , 1}^{M} {obj}_{} + {b}_{J 0 , 1}^{M} {sub}_{} + {b}_{J 45 , 1}^{M} {sub}_{} & Model 2 \\ J {0}_{pred} ( {\tilde{M}}_{obj} , {obj}_{} , {obj}_{} , {sub}_{} , {sub}_{} ) = {a}_{M , 1}^{J 0} {\tilde{M}}_{obj} + {\sum }_{i = 0}^{4} {a}_{J 0 , i}^{J 0} {obj}_{i}^{} + {a}_{J 45 , 1}^{J 0} {obj}_{} + {b}_{M , 1}^{J 0} {\tilde{M}}_{sub} + {b}_{J 45 , 1}^{J 0} {sub}_{} & \\ J {45}_{pred} ( {\tilde{M}}_{obj} , {obj}_{} , {obj}_{} , {sub}_{} , {sub}_{} ) = {a}_{M , 1}^{J 45} {\tilde{M}}_{obj} + {a}_{J 0 , 1}^{J 45} {obj}_{} + {\sum }_{i = 0}^{4} {a}_{J 45 , i}^{J 45} {obj}_{i}^{} + {b}_{M , 1}^{J 45} {\tilde{M}}_{sub} + {b}_{J 0 , 1}^{J 45} {sub}_{} & \end{matrix}$$
where:
(M_{pred}, J0_{pred}, J45_{pred}) denotes the power vector of the predicted refraction;
(\tilde{M}_{obj}, [symbol] _{obj}, [symbol] _{obj}) denotes the power vector of the measurement values from the objective refraction;
(\tilde{M}_{sub}, [symbol] _{sub}, [symbol] _{sub}) denotes the power vector of the measurement values from the subjective refraction;
a_{X,i}^{Y} denote the parameters of the respective parametric function,
Y stands for a power vector component of the power vector of the predicted refraction;
X stands for a power vector component of the power vector of the measured objective refraction.

31. The method according to claim 25, wherein the calculation of the estimated value of the vision disorder of the eye comprises forming a weighted average of the measured values from the first measurement and the second measurement, wherein the first measurement is weighted with first weights and the second measurement is weighted with second weights, wherein optionally, among the first measurement and the second measurement, the measurement having the lower measurement inaccuracy is weighted with higher weights.

32. The method according to claim 31 when dependent on claim 3, wherein the weights are dependent on the measured values of the vision disorder.

33. The method according to claim 32, wherein the measured values comprise an addition and/or a spherical equivalent and the weights are dependent on the addition and/or the difference between the measurement value of the spherical equivalent from the first measurement and the measurement value of the spherical equivalent from the second measurement.

34. The method according to claim 25, wherein:
the measurement inaccuracies or measurement deviations of the first measurement and the second measurement are determined for the object distance Infinite; and/or
the measurement inaccuracies or measurement deviations of the first measurement and the second measurement are determined separately for different apparatuses; and/or
the measurement inaccuracies or measurement deviations of the first measurement and the second measurement are determined at a distance to the eye that is identical for all data.

35. A non-transitory computer program product, which, when loaded into the memory of a computer and executed thereon, causes the computer to carry out a method according to claim 25.

36. A device for determining the vision disorder of an eye of a spectacle wearer with a computing device designed to carry out the method according to claim 25.

37. A method for producing a spectacle lens, comprising:
determining the vision disorder of an eye of the spectacle wearer according to the method according to claim 25;
setting the target power based on the determined vision disorder, so that the target power of the spectacle lens corrects the determined vision disorder in at least one reference point; and
manufacturing the spectacle lens so that the target power is achieved in the at least one predetermined reference point of the spectacle lens.

38. A device for producing a spectacle lens, comprising;
a determining device designed to determine the vision disorder of an eye of a spectacle wearer according to claim 12;
a setting device designed to set the target power in a reference point of the spectacle lens based of the determined vision disorder, and
a manufacturing device designed to manufacture the spectacle lens, so that the target power is achieved in the at least one predetermined reference point of the spectacle lens, preferably in a predetermined wearing position of the spectacle lens.

39. A spectacle lens for correcting the vision disorder of the eye of a spectacle wearer, wherein:
the spectacle lens has a first power P_A in a reference point of the spectacle lens, and
the vision disorder is characterized by at least a first measurement value P_A1 obtained using a measuring device of the first type for measuring the vision disorder and consisting of several components, and at least a second measurement value P_A2 obtained using a measuring device of the second type for measuring the vision disorder and consisting of several components, the first measurement value P_A1 and the second measurement value P_A2 differing in at least one component X,
the component X of the first power P_A present in the reference point of the spectacle lens is closer to the component X of the measurement value among the measurement values P_A1 or P_A2 of the spectacle lens that is obtained from the measuring device with the lower inaccuracy in the measurement of the component X, and
the components of the measurement values P_A1 and P_A2 are components of a wavefront representation of the vision disorder, its linear combination or variables derived therefrom.

40. The spectacle lens according to claim 39, wherein
the component X of the power P_A present in the reference point of the first spectacle lens and the component X of the first measurement value of the first eye P_A1 are substantially identical.

41. A series of spectacle lenses, comprising at least two spectacle lenses A and B according to claim 39 with different powers P_A and P_B in a reference point of the respective spectacle lens for correcting two different vision disorders.

42. A series of spectacle lenses, comprising:
a first spectacle lens A designed to correct a vision disorder of a first eye of a spectacle wearer, wherein the spectacle lens A has a first power P_A in a reference point of the spectacle lens, wherein the vision disorder of the first eye is characterized by at least a first measurement value P_A1 obtained using a measuring device of the first type for measuring the vision disorder and consisting of several components, and at least a second measurement value P_A2 obtained using a measuring device of the second type for measuring the vision disorder and consisting of several components, wherein optionally the first measurement value P_A1 and the second measurement value P_A2 differ in at least one component X;
a second spectacle lens B designed to correct a vision disorder of a second eye of a spectacle wearer, wherein the spectacle lens B has a second power P_B in a reference point identified identically in comparison with the first spectacle lens, wherein the vision disorder of the second eye is characterized by at least a first measurement value P_B1 obtained using a measuring device of the first type and consisting of several components, and at least a second measurement value P_B2 obtained using a measuring device of the second type and consisting of several components, wherein optionally the first measurement value P_B1 and the second measurement value P_B2 differ in at least one component X;
at least a third spectacle lens C designed to correct a vision disorder of a third eye of a spectacle wearer, wherein the spectacle lens C has a third power P_C in a reference point identified identically in comparison with the first spectacle lens, and wherein the vision disorder of the third eye is characterized by at least a first measurement value P_C1 obtained using a measuring device of the first type and consisting of several components, and at least a second measurement value P_C2 obtained using a measuring device of the second type and consisting of several components, wherein optionally the first measurement value P_C1 and the second measurement value P_C2 differ in at least one component X;
wherein:
the first measurement values P_A1, P_B1, and P_C1 determined with the measuring device of the first type are identical in terms of components,
the components X of the second measurement values P_A2, P_B2, and P_C2 determined with the measuring device of the second type all differ pairwise,
the component X of the first power P_A and the component X of the first measurement value P_A1 are substantially identical, and
wherein for the components X of the power of the i^{th} spectacle lens present in the reference point, X_i, where i=A, B or C, and for the components X of the second measurement values of the i^{th} eyes, X_i2, the following relationships apply:
(X_B−X_A)/(X_B2−X_A2) unequal (X_C−X_A)/(X_C2−X_A2);
abs(X_B2−X_A2)<abs(X_C2−X_A2); and
signum(X_B2−X_A2)=signum(X_C2−X_A2), and
wherein the spectacle lenses A, B, and C are single vision lenses or progressive lenses have the same addition.

43. The series of spectacle lenses according to claim 42, wherein
the first, second and third spectacle lenses are single vision lenses or progressive lenses having the same addition Add, where Add<=1.5 dpt, and
for the components X of the power of the i^{th} spectacle lens present in the reference point, X_i, and for the components X of the second measurement values of the i^{th} eyes, X_i2, the following relationships apply:
(X_B−X_A)/(X_B2−X_A2)<(X_C−X_A)/(X_C2−X_A2) if X_B2−X_A2>0,X_C2−X_A2>0,
and
(X_B−X_A)/(X_B2−X_A2)>(X_C−X_A)/(X_C2−X_A2) if X_B2−X_A2<0,X_C2−X A2<0.

44. The series of spectacle lenses according to claim 42, wherein
the first, second and third spectacle lenses are progressive lenses having the same addition Add, where Add<=2 dpt, and
for the components X of the power of the i^{th} spectacle lens present at the reference point, X_i, and for the components X of the second measurement values of the i^{th} eyes, X_i2, the following relationships apply:
(X_B−X_A)/(X_B2−X_A2)>(X_C−X_A)/(X_C2−X_A2) if X_B2−X_A2>0,X_C2−X_A2>0,
and
(X_B−X_A)/(X_B2−X_A2)>(X_C−X_A)/(X_C2−X_A2) if X_B2−X_A2<0,X_C2−X A2<0.

45. The series of spectacle lenses according to claim 41, wherein
the measuring device of the first type is a measuring device for measuring the subjective refraction, and/or
the measuring device of the second type is a measuring device for measuring the objective refraction.

46. The series of spectacle lenses according to claim 41, further comprising:
at least a fourth spectacle lens D designed to correct a vision disorder of a fourth eye of a spectacle wearer, wherein the spectacle lens D has a fourth power P_D at least in a reference point identified identically in comparison with the first spectacle lens, wherein the vision disorder of the fourth eye is characterized by at least a first measurement value P_D1 obtained using a measuring device of the first type and consisting of several components and at least a second measurement value P_D2 obtained using a measuring device of the second type and consisting of several components, wherein the first measurement value P_D1 and the second measurement value P_D2 differ in at least one component X;
at least a fifth spectacle lens E designed to correct a vision disorder of a fifth eye of a spectacle wearer, wherein the spectacle lens E has a fifth power P_E at least in a reference point identified identically in comparison with the first spectacle lens, wherein the vision disorder of the fifth eye is characterized by at least a first measurement value P_E1 obtained using a measuring device of the first type and consisting of several components and at least a second measurement value P_E2 obtained using a measuring device of the second type and consisting of several components, wherein optionally the first measurement value P_E1 and the second measurement value P_E2 differ in at least one component X; and
wherein:
P_A1, P_D1 and P_E1 of the first, fourth, and fifth eyes are identical in terms of components,
the components X of the second measurement values P_A2, P_D2, and P_E2 of the first, fourth, and fifth eyes determined with the measuring device of the second type all differ pairwise,
the component X of the first power P_A present in the reference point of the first spectacle lens and the component X of the first measurement value of the first eye P_A1 are substantially identical, and
for the components X of the power of the i^{th} spectacle lens present in the reference point, X_i, and for the components X of the second measurement values of the i^{th} eyes, X_i2, the following relationships apply:
X_D2−X_A2>0,
X_E2−X_A2<0,
X_D−X_A>0, and
X_E−X_A<0.

47. A method for ordering spectacle lenses, comprising:
providing measurement values from a first measurement and a second measurement of the vision disorder of the eye of the spectacle wearer; and
calculating an estimated value for the vision disorder of the eye of the spectacle wearer based on the measurement values from the first measurement and the second measurement, wherein measurement inaccuracies or measurement deviations of the first measurement and the second measurement of the vision disorder are taken into account in the calculation of the estimated value of the vision disorder.

48. A device for ordering spectacle lenses, comprising:
a device designed to provide measurement values from a first measurement and a second measurement of the vision disorder of the eye of the spectacle wearer; and
a computing device designed to calculate an estimated value for the vision disorder of the eye of the spectacle wearer based on the measurement values from the first measurement and the second measurement, wherein measurement inaccuracies of the first measurement and the second measurement of the vision disorder are taken into account in the calculation of the estimated value of the vision disorder.
